(19) United States
(12) Patent Application Publication
(10) Pub. No.: US 2026/0232930 A1
(43) Pub. Date: Aug. 13, 2026

(54) SUCTION DEVICE AND INFORMATION PROCESSING METHOD

(71) Applicant: Japan Tobacco Inc., Tokyo (JP)

(72) Inventors: Takashi FUJIKI, Tokyo (JP); Ryo YOSHIDA, Tokyo (JP); Satoshi NAKAMURA, Tokyo (JP)

(73) Assignee: Japan Tobacco Inc., Tokyo (JP)

(21) Appl. No.: 19/136,360

(22) PCT Filed: Dec. 16, 2022
(86) PCT No.: PCT/JP2022/046322

§ 371 Date: Jun. 6, 2025

(51) Int. Cl. A61M 15/00; A61M 11/04; G16H 40/63

(52) U.S. Cl. CPC A61M 15/0001; A61M 11/041; G16H 40/63; A61M 2205/14; A61M 2205/3306; A61M 2205/502; A61M 2205/8212

(57) ABSTRACT

Provided is a suction device provided with: a housing section having an inner space and an opening; a first detection unit which detects the state of the inner space; a second detection unit which detects information associated with the state of the suction device; and a control unit that performs, on the basis of a detection result obtained by the second detection unit, the control of the switching of the mode of the first detection unit to an operation mode or a stop mode and the determination whether or not the control of the operation of the suction device is performed in accordance with a detection value detected by the first detection unit when the mode of the first detection unit is in the operation mode.

### TECHNICAL FIELD

The present disclosure relates to an inhalation device and an information processing method.

### BACKGROUND ART

Inhalation devices that generate substances to be inhaled by a user, such as e-cigarettes and nebulizers, are in widespread use. For example, an inhalation device employs an aerosol source for generating an aerosol, and a substrate including a flavor source or the like for imparting a flavor component to the generated aerosol, to generate an aerosol to which the flavor component has been imparted. The user can enjoy the flavor by inhaling the aerosol to which the flavor component has been imparted, generated by the inhalation device. The action by which the user inhales the aerosol is also referred to below as “puffing” or a “puffing action”.

Various technical developments are underway for the purpose of further improving the quality of a user experience when using such an inhalation device. For example, PTL 1 below describes technology in which light is emitted, a phosphorescence characteristic of reflected light is detected, and operation of an inhalation device is controlled on the basis of the detection result.

### CITATION LIST

### Patent Literature

PTL 1: JP 2019-528710 A

### SUMMARY OF INVENTION

### Technical Problem

However, controlling operation of an inhalation device on the basis of detection results from a sensor could also feasibly be contrary to the user's expectations, depending on the state of the inhalation device.

Accordingly, the present disclosure takes account of the abovementioned problem, and one objective of the present disclosure lies in providing a mechanism capable of further improving the quality of the user experience.

### Solution to Problem

One aspect of the present disclosure for solving the problem above provides an inhalation device comprising: an accommodating portion having an internal space and an opening enabling the internal space to communicate with the outside; a first detection unit for detecting a state of the internal space; a second detection unit for detecting information relating to a state of the inhalation device; and a control unit for performing, based on a detection result obtained by means of the second detection unit: control to switch a mode of the first detection unit to an operating mode for detecting the state of the internal space, or a stop mode for stopping detection of the state of the internal space; and, when the mode of the first detection unit is the operating mode, a decision of whether or not to control an operation of the inhalation device in accordance with a detection value detected by means of the first detection unit.

The inhalation device may further comprise a heating unit for heating a substrate accommodated in the accommodating portion, and the operation of the inhalation device, for which the decision of whether or not to perform control, which is made by the control unit based on the detection result obtained by means of the second detection unit, may be heating afforded by the heating unit.

The inhalation device may further comprise a cover portion capable of opening/closing the opening leading to the internal space of the accommodating portion, and the second detection unit may detect at least any of: opening/closing of the opening by the cover portion, input of an instruction to start or stop detection by the first detection unit, or an automatic resolution-impossible error state, which is a state where an error relating to operation of the inhalation device has occurred, and the error cannot be automatically resolved by means of the control unit.

The control unit may perform control to switch the mode of the first detection unit from the operating mode to the stop mode when the second detection unit has detected closure of the opening, input of an instruction to stop detection by the first detection unit, or an automatic resolution-impossible error state.

The inhalation device may comprise a plurality of first detection units, and the control unit may perform control to switch the mode of all of the plurality of first detection units which are in the operating mode to the stop mode when the second detection unit has detected closure of the opening, input of an instruction to stop detection by the first detection units, or an automatic resolution-impossible error state.

The control unit may perform control to switch the mode of the first detection units from the stop mode to the operating mode when the second detection unit has detected opening of the opening, input of an instruction to start detection by the first detection units, or clearing of an automatic resolution-impossible error state.

The control unit may perform control to switch the mode of only one of the plurality of first detection units from the stop mode to the operating mode when the second detection unit has detected opening of the opening, input of an instruction to start detection by the first detection unit, or clearing of an automatic resolution-impossible error state.

The inhalation device may further comprise: a heating unit for heating a substrate accommodated in the accommodating portion; and a power source unit for storing power, and the second detection unit may detect at least any of: connection and disconnection of charging of the power source unit; input of an instruction to shift to a state in which heating by the heating unit can be implemented or is prohibited; the start or termination of switching, by means of the control unit, of a heating profile indicating a time-series transition of heating performed by the heating unit; the start or clearing of an automatic resolution error state, which is a state where an error relating to operation of the inhalation device has occurred, and the error can be automatically resolved by means of the control unit; or an instruction to cause the inhalation device to sleep or to cancel sleeping.

Connection of charging of the power source unit, input of an instruction to shift to a state in which heating by the heating unit is prohibited, the start of switching of the heating profile by means of the control unit, the start of an automatic resolution error state, or an instruction to cause the inhalation device to sleep, may constitute a first operation; disconnection of charging of the power source unit, input of an instruction to shift to a state in which heating by the heating unit can be implemented, termination of switching of the heating profile by means of the control unit, clearing of an automatic resolution error state, or an instruction to cancel sleeping of the inhalation device, may constitute a second operation; and when the first operation has been detected by the second detection unit, the control unit may decide that control of operation of the inhalation device based on the detection value detected by the first detection unit should not be performed until the second operation is detected.

The inhalation device may comprise a plurality of first detection units, and the control unit may control the plurality of first detection units so that the mode of only one of the plurality of first detection units is the operating mode when the first operation has been detected by the second detection unit.

When the first operation has been detected by means of the second detection unit during a period from detection by the first detection unit of a detection value exceeding a first threshold for determining insertion of the substrate until detection of a detection value falling below a second threshold for determining withdrawal of the substrate, the control unit may decide that control of operation of the inhalation device based on the detection value detected by means of the first detection unit should not be performed until a detection value falling below the second threshold is detected by means of the first detection unit.

When a detection value exceeding the first threshold has been detected during a period from detection of the first operation by the second detection unit until detection of the second operation by the second detection unit, the control unit may decide that control of operation of the inhalation device based on the detection value detected by means of the first detection unit should not be performed until a detection value falling below the second threshold is detected by means of the first detection unit.

The first detection unit may detect the state of the internal space by emitting light into the internal space and detecting reflected light received.

The inhalation device may further comprise a substrate accommodated in the accommodating portion.

Furthermore, another aspect of the present disclosure for solving the problem above provides an information processing method implemented by means of a computer for controlling an inhalation device comprising: an accommodating portion having an internal space and an opening enabling the internal space to communicate with the outside; a first detection unit for detecting a state of the internal space; and a second detection unit for detecting information relating to a situation of the inhalation device, and the information processing method comprises performing, based on a detection result obtained by means of the second detection unit: control to switch a mode of the first detection unit to an operating mode for detecting the state of the internal space, or a stop mode for stopping detection of the state of the internal space; and, when the mode of the first detection unit is the operating mode, a decision of whether or not to control an operation of the inhalation device in accordance with a detection value detected by means of the first detection unit.

### Advantageous Effects of Invention

The present disclosure as described above provides a mechanism capable of further improving the quality of a user experience.

### BRIEF DESCRIPTION OF DRAWINGS

FIG. 1 is a schematic diagram illustrating schematically an internal configuration example of an inhalation device.

FIG. 2 is an overall oblique view of an inhalation device 100 according to the embodiment.

FIG. 3 is an overall oblique view of the inhalation device 100 according to the embodiment, with a stick-type substrate 150 held therein.

FIG. 4 schematically shows a configuration close to an accommodating portion 140 of the inhalation device 100 according to the embodiment.

FIG. 5 is a schematic diagram showing a detailed configuration close to a light sensor unit 170 of the inhalation device 100 according to the embodiment.

FIG. 6 is a schematic diagram in which the accommodating portion 140 of the inhalation device 100 according to the embodiment is seen from an opening 142 side (i.e., from the top).

FIG. 7 is a block diagram showing a configuration of the light sensor unit 170 of the inhalation device 100 according to the embodiment.

FIG. 8 shows an example of operation of the light sensor unit 170 on a time axis.

FIG. 9 is an explanatory diagram to illustrate a specific example of a detection control unit 179 sending an interrupt notification.

FIG. 10 shows an example of a configuration of a cleaning article 190 according to the embodiment.

FIG. 11 schematically shows a situation in which the accommodating portion 140 having the stick-type substrate 150 inserted therein is seen from the opening 142 side (i.e., from the top).

FIG. 12 schematically shows a situation in which the accommodating portion 140 having the cleaning article 190 inserted therein is seen from the opening 142 side (i.e., from the top).

FIG. 13 is a flowchart showing an example of a control processing flow for automatic heating executed by the inhalation device 100 according to the embodiment.

FIG. 14 is a flowchart illustrating an example of a determination processing flow for an inserted article based on multiple substitution control operations, which is executed by the inhalation device 100 according to the embodiment.

FIG. 15 is a flowchart illustrating an example of a control processing flow for automatic heating in accordance with a detection result of a sensor unit 112, which is executed by the inhalation device 100 according to the embodiment.

FIG. 16 is a flowchart showing an example of a control processing flow for resetting an automatic heating control flow, which is executed by the inhalation device 100 according to the embodiment.

### DESCRIPTION OF EMBODIMENTS

Preferred embodiments of the present disclosure will be described in detail below with reference to the appended drawings. It should be noted that components having substantially the same functional configuration will be assigned the same reference numbers in the description and drawings to avoid giving a duplicate description.

In this description and the drawings, elements having substantially identical functional configurations may also be distinguished by using the same reference sign followed by a different letter of the alphabet. For example, a plurality of elements having a substantially identical functional configuration are distinguished as a “light sensor unit 170A” and a “light sensor unit 170B”. However, if there is no need to specifically distinguish between each of the plurality of elements having a substantially identical functional configuration, only the same reference sign is assigned. For example, if there is no need to distinguish between the light sensor unit 170A and the light sensor unit 170B, these are simply referred to as the “light sensor unit(s) 170”.

### 1. Configuration Example of Inhalation Device

### (1) Internal Configuration Example

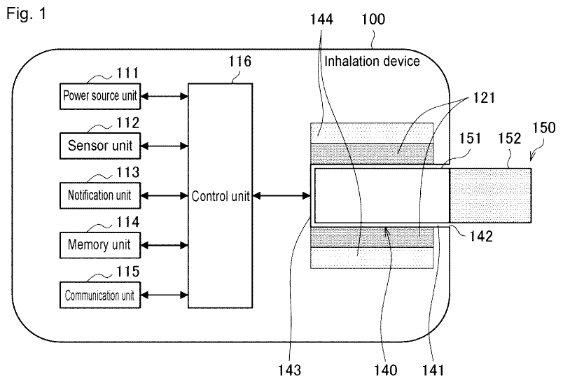

FIG. 1 is a schematic diagram illustrating schematically an internal configuration example of an inhalation device. As illustrated in FIG. 1, an inhalation device 100 according to the present configuration example comprises a power source unit 111, a sensor unit 112, a notification unit 113, a memory unit 114, a communication unit 115, a control unit 116, a heating unit 121, an accommodating portion 140, and a heat insulating portion 144.

The power source unit 111 stores electrical power. The power source unit 111 then supplies the electric power to each component of the inhalation device 100 in accordance with control performed by the control unit 116. The power source unit 111 may be configured, for example, by a rechargeable battery such as a lithium ion secondary battery.

The sensor unit 112 acquires various types of information relating to the inhalation device 100. As an example, the sensor unit 112 is configured by a pressure sensor such as a condenser microphone, a flow rate sensor or a temperature sensor, etc., and acquires values associated with inhalation by a user. As another example, the sensor unit 112 is configured by an input device, such as a button or switch, for accepting input of information from the user.

The notification unit 113 notifies the user of information. The notification unit 113 is configured by a light-emitting device which emits light, a display device which displays images, a sound output device which outputs sound, or a vibration device which vibrates, etc., for example.

The memory unit 114 stores various types of information for the operation of the inhalation device 100. The memory unit 114 is configured by a non-volatile storage medium such as a flash memory, for example.

The communication unit 115 is a communication interface capable of performing communication conforming to any wired or wireless communication standard. Examples of communication standards that may be used include standards that employ Wi-Fi (registered trademark), Bluetooth (registered trademark), BLE (Bluetooth Low Energy) (registered trademark), NFC (Near-Field Communication), or LPWA (Low Power Wide Area), for example.

The control unit 116 functions as an arithmetic processing device and a control device, and controls overall operation within the inhalation device 100 in accordance with various programs. The control unit 116 is realized by a CPU (Central Processing Unit) or an electronic circuit such as a microprocessor, for example.

The accommodating portion 140 has an internal space 141, and holds a stick-type substrate 150 while accommodating a portion of the stick-type substrate 150 in the internal space 141. The accommodating portion 140 has an opening 142 allowing the internal space 141 to communicate with the outside, and accommodates the stick-type substrate 150 that has been inserted into the internal space 141 from the opening 142. For example, the accommodating portion 140 is a cylindrical body comprising the opening 142 and a bottom portion 143 serving as a bottom surface, and defines a columnar internal space 141. An air flow path for supplying air to the internal space 141 is connected to the accommodating portion 140. An air inflow hole, which is an inlet for air into the air flow path, is disposed in a side surface of the inhalation device 100, for example. An air outflow hole serving as an outlet for air from the air flow path to the internal space 141 is disposed in the bottom portion 143, for example.

The stick-type substrate 150 comprises a substrate portion 151 and a mouthpiece portion 152. The substrate portion 151 contains an aerosol source. The aerosol source includes a tobacco-derived or non-tobacco-derived flavor component. If the inhalation device 100 is a medical inhaler such as a nebulizer, the aerosol source may include a drug. The aerosol source may, for example, be a liquid such as water or a polyhydric alcohol, for example glycerol or propylene glycol, containing the tobacco-derived or non-tobacco-derived flavor component, or may be a solid including the tobacco-derived or non-tobacco-derived flavor component. In a state in which the stick-type substrate 150 is being held in the accommodating portion 140, at least a portion of the substrate portion 151 is accommodated in the internal space 141, and at least a portion of the mouthpiece portion 152 protrudes from the opening 142. Then, when the user holds the mouthpiece portion 152 protruding from the opening 142 in their mouth and inhales, air flows into the internal space 141 via the air flow path, which is not illustrated in the drawings, and reaches the inside of the user's mouth together with the aerosol generated from the substrate portion 151.

The heating unit 121 heats the aerosol source to atomize the aerosol source, thereby generating the aerosol. In the example shown in FIG. 1, the heating unit 121 has a film-like form and is arranged so as to cover an outer circumference of the accommodating portion 140. Then, when the heating unit 121 generates heat, the substrate portion 151 of the stick-type substrate 150 is heated from the outer circumference and an aerosol is generated. The heating unit 121 generates heat when supplied with electricity from the power source unit 111. By way of example, electricity may be supplied when the sensor unit 112 detects that the user has started sucking and/or that predetermined information has been input. The supply of electricity may then be stopped when the sensor unit 112 detects that the user has finished sucking and/or that predetermined information has been input.

The heat insulating portion 144 prevents heat transfer from the heating unit 121 to other components. For example, the heat insulating portion 144 is configured from a vacuum heat insulating material or an aerogel heat insulating material, or the like.

A configuration example of the inhalation device 100 has been described above. The inhalation device 100 is, of course, not limited to the configuration described above, and may adopt various configurations, such as those illustrated below by way of example.

As one example, the heating unit 121 may have a blade-like form and may be arranged so as to protrude into the internal space 141 from the bottom portion 143 of the accommodating portion 140. In that case, the blade-like heating unit 121 is inserted into the substrate portion 151 of the stick-type substrate 150 and heats the substrate portion 151 of the stick-type substrate 150 from the inside. As another example, the heating unit 121 may be arranged so as to cover the bottom portion 143 of the accommodating portion 140. Furthermore, the heating unit 121 may be configured by a combination of two or more from among a first heating unit covering the outer circumference of the accommodating portion 140, a blade-like second heating unit, and a third heating unit covering the bottom portion 143 of the accommodating portion 140.

As another example, the accommodating portion 140 may comprise an opening/closing mechanism such as a hinge for opening/closing part of a casing that forms the internal space 141. By opening/closing the casing, the accommodating portion 140 may then receive and grip the stick-type substrate 150 which has been inserted into the internal space 141. In that case, the heating unit 121 may be provided on the part of the accommodating portion 140 gripping the stick-type substrate 150, and may heat the stick-type substrate 150 while pressing same.

Furthermore, the means for atomizing the aerosol source is not limited to heating provided by the heating unit 121. For example, the means for atomizing the aerosol source may be induction heating.

It may be understood that an aerosol-generating system for generating an aerosol is constructed by collaboration of the inhalation device 100 and the stick-type substrate 150. Alternatively, the inhalation device 100 may be understood to comprise the stick-type substrate 150.

### (2) Exterior Configuration Example

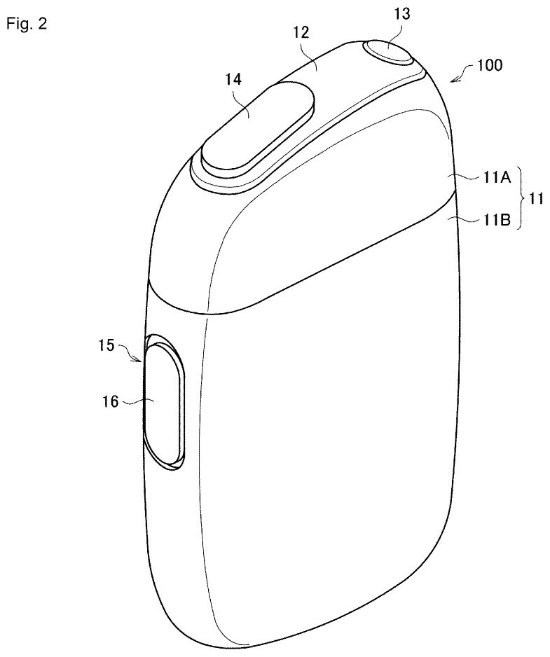

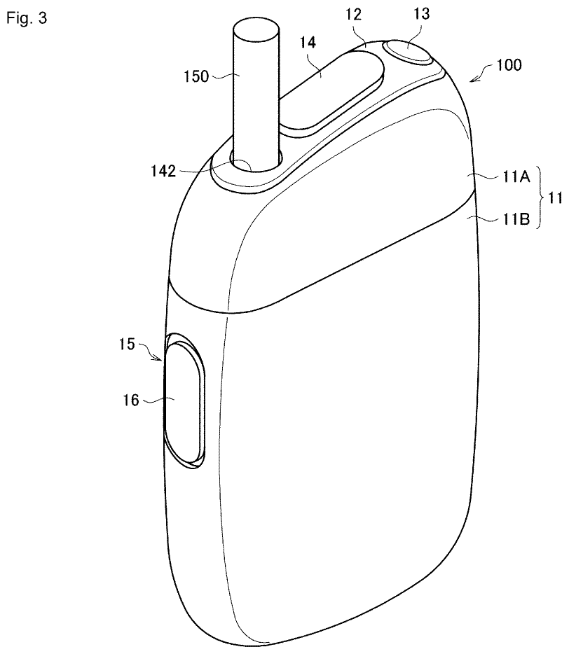

FIG. 2 is an overall oblique view of the inhalation device 100 according to the embodiment. FIG. 3 is an overall oblique view of the inhalation device 100 according to the embodiment, with the stick-type substrate 150 held therein.

As shown in FIGS. 2 and 3, the inhalation device 100 comprises: a top housing 11A, a bottom housing 11B, a cover 12, a switch 13, a cover portion 14, a ventilation port 15, and a cap 16. The top housing 11A and the bottom housing 11B are connected to each other to thereby construct an outermost outer housing 11 of the inhalation device 100. The outer housing 11 is of a size that fits in a user's hand. When the user is using the inhalation device 100, the user can inhale a flavor while holding the inhalation device 100 in their hand.

The top housing 11A has an opening which is not depicted, and the cover 12 is joined to the top housing 11A to close this opening. As shown in FIG. 3, the cover 12 comprises an opening 142 enabling insertion of the stick-type substrate 150. The cover portion 14 is configured to open/close the opening 142 in the cover 12.

The switch 13 is used to switch the operation of the inhalation device 100 on and off. For example, in a state in which the stick-type substrate 150 has been inserted into the internal space 141 from the opening 142, as shown in FIG. 3, the user operates the switch 13 whereby power is supplied from the power source unit 111 to the heating unit 121, and the stick-type substrate 150 can be heated without being burned. When the stick-type substrate 150 is heated, an aerosol is generated from the aerosol source contained in the stick-type substrate 150, and the flavor of the flavor source is taken in by the aerosol. The user then draws on the part of the stick-type substrate 150 protruding from the inhalation device 100 (the part depicted in FIG. 3, i.e., the mouthpiece portion 152), and the user can thereby inhale the aerosol containing the flavor.

The ventilation port 15 is a ventilation port for introducing air into the internal space 141. The air taken inside the inhalation device 100 from the ventilation port 15 is introduced into the internal space 141 from the bottom portion 143 of the accommodating portion 140, for example. The cap 16 is detachable from the bottom housing 11B. The ventilation port 15 is formed between the bottom housing 11B and the cap 16 by attaching the cap 16 to the bottom housing 11B. The cap 16 may have a through-hole or a cutout, etc. which is not depicted, for example.

### 2. Technical Features

### (1) Detailed Configuration Close to Accommodating Portion 140

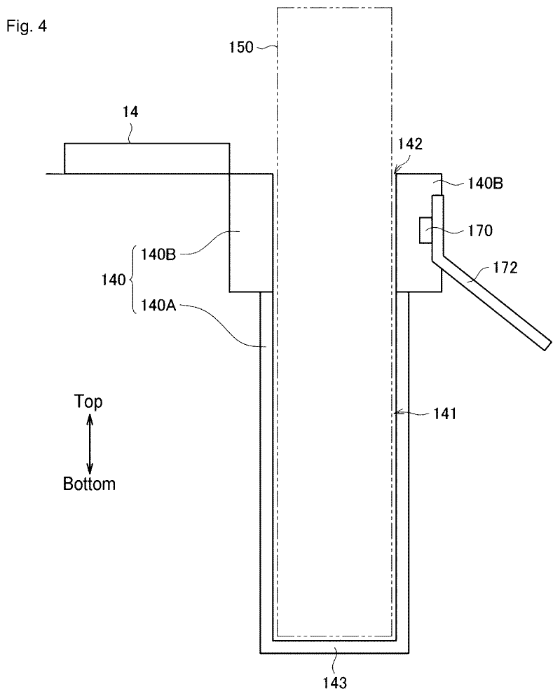

FIG. 4 schematically shows a configuration close to the accommodating portion 140 of the inhalation device 100 according to the embodiment. FIG. 4 schematically shows a state in which the stick-type substrate 150 is accommodated in the accommodating portion 140. As shown in FIG. 4, the inhalation device 100 comprises: the cover portion 14, a stick lower portion accommodating portion 140A, a guide portion 140B, the opening 142, the bottom portion 143, a light sensor unit 170, and a circuit board 172. The direction of insertion/withdrawal of the stick-type substrate 150 into/from the inhalation device 100 will also be referred to below as the vertical direction. The direction in which the stick-type substrate 150 is inserted will also be referred to as “down” or “bottom”, etc., and the direction in which the stick-type substrate 150 is withdrawn will also be referred to as “up” or “top”, etc.

The stick lower portion accommodating portion 140A is a bottomed cylindrical body constituting the bottom portion 143-side portion of the accommodating portion 140. The stick lower portion accommodating portion 140A accommodates the bottom portion 143-side portion of the stick-type substrate 150 inserted into the internal space 141 from the opening 142.

The guide portion 140B is a cylindrical body which is open at both ends and constitutes the opening 142-side portion of the accommodating portion 140. The guide portion 140B accommodates the part of the stick-type substrate 150 inserted into the internal space 141 from the opening 142 which is accommodated in the accommodating portion 140 but is not accommodated in the stick lower portion accommodating portion 140A. The guide portion 140B also functions as a guide for facilitating insertion of the stick-type substrate 150 into the stick lower portion accommodating portion 140A. For example, the guide portion 140B may be formed with a larger opening diameter than the stick lower portion accommodating portion 140A, or may be formed in the shape of a funnel which gradually decreases in opening diameter from the top toward the bottom.

The light sensor unit 170 emits light into the internal space 141 and detects reflected light received. The light sensor unit 170 is an example of a detection unit in this embodiment and is included in the sensor unit 112. The light sensor unit 170 is an IC (integrated circuit) equipped with an infrared proximity sensor, for example. In this case, the light sensor unit 170 emits infrared radiation into the internal space 141 and detects the infrared radiation reflected by a detected object such as an article accommodated in the internal space 141 or the inner wall of the accommodating portion 140.

The light sensor unit 170 is disposed at a location enabling light to be emitted into the internal space 141. The light sensor unit 170 is disposed in the guide portion 140B, for example. Specifically, the light sensor unit 170 is embedded in the guide portion 140B. The light sensor unit 170 then detects the light reflected by a detected object such as an article accommodated in the internal space 141 or the inner wall of the guide portion 140B.

Here, the heating unit 121 is arranged so as to cover the outer circumference of the stick lower portion accommodating portion 140A. Meanwhile, the heating unit 121 is not arranged on the outer circumference of the guide portion 140B. In addition, the guide portion 140B may be formed by a material having lower thermal conductivity than the material constituting the stick lower portion accommodating portion 140A. The light sensor unit 170 is therefore capable of detecting light without being affected by heating of the stick-type substrate 150.

Furthermore, the inner wall of the guide portion 140B may be black. The guide portion 140B having a black inner wall makes it possible to suppress reflection of the light emitted by the light sensor unit 170. Considering that the stick-type substrate 150 may be formed with a color which reflects light relatively easily, such as white, then it is possible to create a large difference in reflected light intensity when the stick-type substrate 150 is inserted and when it is not inserted.

The circuit board 172 is a board on which the light sensor unit 170 is mounted. The circuit board 172 is an FPC (flexible printed circuit), for example. The circuit board 172 is connected to the control unit 116 by means of a connector or solder, for example.

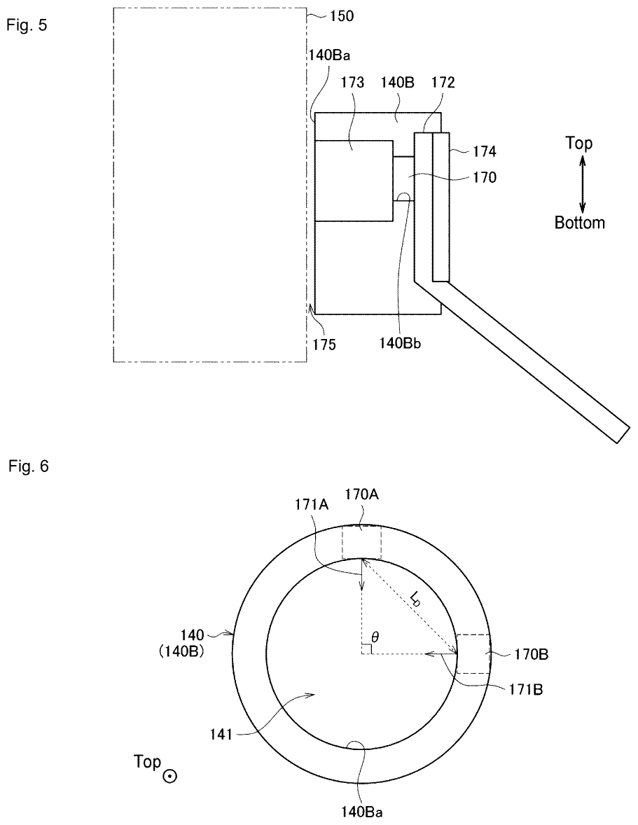

FIG. 5 is a schematic diagram showing a detailed configuration close to the light sensor unit 170 of the inhalation device 100 according to the embodiment. As shown in FIG. 5, the inhalation device 100 further comprises a light transmitting filter 173 and a reinforcing plate 174.

The light transmitting filter 173 is a filter for transmitting the light emitted by the light sensor unit 170. The light transmitting filter 173 is an infrared transmitting filter when the light sensor unit 170 is an infrared proximity sensor, for example. There is no particular limitation as to the material of the light transmitting filter 173, and it may be a resin or glass, or may be a transparent resin having a light transmitting coating. The light transmitting filter 173 may be colored. A colored light transmitting filter 173 makes it possible to conceal the light sensor unit 170 from the outside. A hole 140Bb is provided in an inner wall 140Ba of the guide portion 140B, and the light sensor unit 170 is embedded in this hole 140Bb. The light transmitting filter 173 is arranged so as to close off this hole 140Bb, and forms the inner wall 140Ba of the guide portion 140B. This configuration enables the inner wall 140Ba of the guide portion 140B to be made smoother. Furthermore, the light transmitting filter 173 is capable of maintaining airtightness so that side stream smoke, etc. flowing in from outside the stick does not touch the light sensor unit 170.

A clearance 175 constitutes a gap provided between the stick-type substrate 150 accommodated in the accommodating portion 140, and the inner wall 140Ba of the guide portion 140B. The clearance 175 may be provided so that a distance between the stick-type substrate 150 and the inner wall 140Ba of the guide portion 140B is 1-2 mm.

The reinforcing plate 174 is a plate-like member having a predetermined rigidity. The reinforcing plate 174 is arranged so as to cover a rear side of the circuit board 172 having the light sensor unit 170 disposed on a front side thereof, and reinforces the light sensor unit 170 and the circuit board 172.

FIG. 6 is a schematic diagram in which the accommodating portion 140 of the inhalation device 100 according to the embodiment is seen from the opening 142 side (i.e., from the top). As shown in FIG. 6, the inhalation device 100 may comprise two light sensor units 170 (170A and 170B). The light sensor unit 170A and the light sensor unit 170B are examples of a first detection unit, a first state detection unit, and a second state detection unit. The light sensor unit 170A and the light sensor unit 170B are arranged at an interval, with a distance therebetween being LD. A direction 171A in which light is emitted by the light sensor unit 170A (this will also be referred to below as an emission direction 171A) and a direction 171B in which light is emitted by the light sensor unit 170B (this will also be referred to below as an emission direction 171B) form an angle θ on a plane orthogonal to the vertical direction. Determinations relating to an article inserted into the accommodating portion 140 can be made more accurately by virtue of the fact that the inhalation device 100 comprises a plurality of light sensor units 170 which are also provided at a suitable distance LD and a suitable angle θ. Determination processing employing the light sensor unit 170 will be described in detail later.

### (2) Configuration of Light Sensor Unit 170

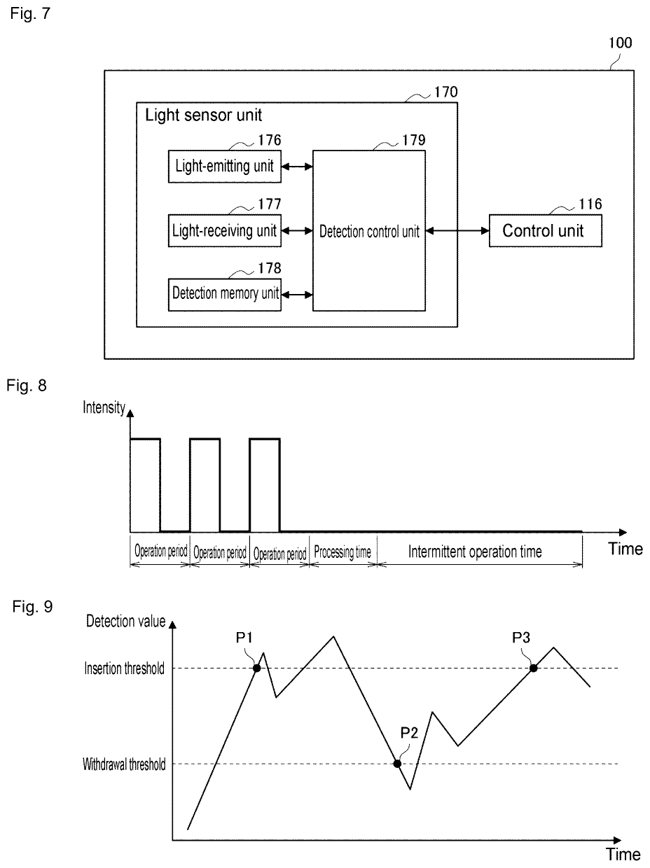

The configuration of the light sensor unit 170 will be described in detail next with reference to FIG. 7. FIG. 7 is a block diagram showing the configuration of the light sensor unit 170 of the inhalation device 100 according to the embodiment.

As shown in FIG. 7, the light sensor unit 170 comprises a light-emitting unit 176, a light-receiving unit 177, a detection memory unit 178, and a detection control unit 179. The light sensor unit 170 is then connected to the control unit 116. The light sensor unit 170 operates under control by the control unit 116.

The light-emitting unit 176 emits light into the internal space 141. The light emitting unit 176 is configured by a light-emitting element such as an LD (laser diode) or an LED (light-emitting diode). The light-emitting unit 176 is an infrared LD which emits infrared radiation in this embodiment. The light-receiving unit 177 detects reflected light from the light emitted by the light-emitting unit 176. The infrared radiation emitted by the light-emitting unit 176 may be a VCSEL (vertical-cavity surface-emitting laser). Operation of the light-emitting unit 176 will be described in detail with reference to FIG. 8.

FIG. 8 shows an example of operation of the light sensor unit 170 on a time axis. The horizontal axis in FIG. 8 shows time passing from left to right. The vertical axis in FIG. 8 shows the intensity of light emitted by the light-emitting unit 176. As shown in FIG. 8, the light-emitting unit 176 emits pulsed light with a predetermined period. This period is also referred to as an operation period. The light-emitting unit 176 repeats pulsed light emission three times and then stops light emission for a processing time and an intermittent operation time. The processing time is the time during which processing based on reflected light detected by means of the light-receiving unit 177 is implemented. The intermittent operation time is the time until the next pulsed light emission. The light-emitting unit 176 repeats the series of operations including pulsed light emission and stopping of light emission described with reference to FIG. 8.

The detection control unit 179 controls operation of components of the light sensor unit 170. An example of the processing implemented by the detection control unit 179 will be described below. This processing is essentially implemented during the processing time described with reference to FIG. 8.

As an example, the detection control unit 179 calculates a value indicating the intensity of reflected light detected by means of the light-receiving unit 177. The calculated value indicating the intensity of reflected light will also be referred to below as a detection value. The detection value calculated by the detection control unit 179 becomes greater as the intensity of the reflected light detected increases. The intensity of the reflected light and the detection value may have a linear relationship.

As another example, the detection control unit 179 may calculate, based on the detection value, the distance to the detected object which reflected the light emitted from the light sensor unit 170, i.e., the distance between the detected object and the light sensor unit 170. More specifically, the distance calculated by the detection control unit 179 becomes shorter as the detection value becomes greater, i.e., as the intensity of the reflected light increases. On the other hand, the distance calculated by the detection control unit 179 becomes longer as the detection value becomes smaller, i.e., as the intensity of the reflected light decreases.

As another example, the detection control unit 179 controls operation of the light-emitting unit 176. More specifically, the detection control unit 179 may control at least any one of the number of times of pulsed light emission, operation period or intermittent operation time shown in FIG. 8. Furthermore, the detection control unit 179 may control the intensity of infrared radiation emitted by means of the light-emitting unit 176 by controlling the value of a current applied to the light-emitting unit 176 (this value will also be referred to below as the LD current value).

As another example, the detection control unit 179 notifies the control unit 116 of information. For example, the detection control unit 179 may cause the detection memory unit 178 to store calculated detection values. If the detection value exceeds or falls below a predetermined threshold, the detection control unit 179 may provide the control unit 116 with a notification to that effect. This notification will also be referred to below as an interrupt notification. In this case, reception of the interrupt notification triggers the control unit 116 to read out the detection value stored in the detection memory unit 178. Additionally, the detection control unit 179 may provide the interrupt notification to the control unit 116 with the calculated detection value included in the notification. The processing relating to such a detection value may also be carried out in the same way for the distance to a detected object. That is, the detection control unit 179 may notify the control unit 116 of the calculated distance. Alternatively, the detection control unit 179 may notify the control unit 116 if the calculated distance exceeds or falls below a predetermined threshold, while the calculated distance has also been stored in the detection memory unit 178.

Moreover, the interrupt notification may be a notification indicating that some kind of article has been inserted into or withdrawn from the accommodating portion 140. In this case, reception of the interrupt notification triggers the control unit 116 to implement predetermined processing. Examples of the predetermined processing may include determining whether or not a stick determination condition (to be described later) has been satisfied, and heating control based on a determination result, etc. By virtue of this configuration, the predetermined processing is implemented only when an interrupt notification has been received, so it is possible to lighten the processing load on the control unit 116.

To give a more specific example, if the calculated detection value exceeds an insertion threshold, which is a predetermined threshold, the detection control unit 179 may send an interrupt notification indicating that some kind of article has been inserted into the accommodating portion 140. An interrupt notification such as this will also be referred to below as a detection interrupt notification. Furthermore, the detection control unit 179 may send an interrupt notification if the calculated detection value falls below a withdrawal threshold, which is a predetermined threshold. An interrupt notification such as this will also be referred to below as a detection-deactivation interrupt notification.

Here, the detection interrupt notification may be sent when a detection value exceeding the insertion threshold is calculated for the first time after a detection value falling below the withdrawal threshold was calculated by means of the detection control unit 179. Furthermore, the detection-deactivation interrupt notification may be sent when a detection value falling below the withdrawal threshold is calculated for the first time after a detection value exceeding the insertion threshold was calculated by means of the detection control unit 179.

In addition, the detection control unit 179 may update an insertion status managed by (i.e., stored in) the detection memory unit 178, at the same time as sending the interrupt notification. The insertion status indicates a state of insertion or non-insertion of an article in the accommodating portion 140. The detection control unit 179 may update the insertion status to “article inserted” at the same time as sending a detection interrupt notification. Furthermore, the detection control unit 179 may update the insertion status to “article not inserted” at the same time as sending a detection-deactivation interrupt notification. When the insertion status is managed by the detection memory unit 178, the detection control unit 179 may send an interrupt notification without distinguishing between a detection interrupt notification and a detection-deactivation interrupt notification. Reception of the interrupt notification may then trigger the control unit 116 to read out the insertion status stored in the detection memory unit 178.

A specific example of sending of the interrupt notification by the detection control unit 179 will be described here with reference to FIG. 9. FIG. 9 is an explanatory diagram to illustrate a specific example of the detection control unit 179 sending an interrupt notification. The horizontal axis in FIG. 9 shows time passing from left to right. The vertical axis in FIG. 9 shows detection values calculated by the detection control unit 179. That is to say, FIG. 9 shows temporal changes in detection values. After a detection value falling below the withdrawal threshold was detected, the detection control unit 179 detects a detection value exceeding the insertion threshold at a detection point P1. The detection control unit 179 therefore sends an interrupt notification at the detection point P1 and updates the insertion status to “article inserted”.

The detection control unit 179 then detects a detection value falling below the withdrawal threshold at a detection point P2. The detection control unit 179 therefore sends an interrupt notification at the detection point P2 and updates the insertion status to “article not inserted”. Following this, the detection control unit 179 once again detects a detection value exceeding the insertion threshold at a detection point P3. The detection control unit 179 therefore sends an interrupt notification at the detection point P3 and updates the insertion status to “article inserted”.

By using two thresholds (insertion threshold and withdrawal threshold), it is possible to accurately determine whether or not an article is inserted. To be more specific, detection values may fluctuate up and down due to the effects of external interference, noise of the power source supplied to the light sensor unit 170, variations in the shape of the inserted article, variations in the distance between the inserted article and the light-emitting unit 176 and light-receiving unit 177 caused by contact between the user and the inserted article or the user placing the inserted article in their mouth, temperature drift of detection values of the detection control unit 179 caused by changes in ambient temperature, or variations in the rolled diameter of the stick-type substrate 150 (inserted article) caused by puffs during smoking, etc. Even in such cases, it is possible to ensure that the insertion status does not change unless there is a large fluctuation above or below both the insertion threshold and the withdrawal threshold. It is therefore possible to prevent a situation where the result of determining insertion or non-insertion frequently varies, and it is possible to accurately determine whether or not an article is inserted as a result.

As another example, the detection control unit 179 may perform calibration. Specifically, the detection control unit 179 may adjust a relationship between the intensity of reflected light detected by means of the light-receiving unit 177 and the calculated detection value, so that the same detection value is calculated under predetermined conditions. By performing calibration, it is possible to exclude deviations in detection values caused by temperature or vibration, etc. and to exclude effects such as deterioration over time of the light-emitting unit 176 or the light-receiving unit 177.

It should be noted that FIG. 8 shows an example in which pulsed light is emitted three times by the light-emitting unit 176, but there is no particular limitation as to the number of times of pulsed light emission. Furthermore, when pulsed light is emitted multiple times by the light-emitting unit 176, the detection control unit 179 may perform processing by using detection results received multiple times by the light-receiving unit 177, or may perform processing by using some of the detection results received multiple times by the light-receiving unit 177.

The detection memory unit 178 stores programs executed by the detection control unit 179 and various types of data, etc. The detection memory unit 178 may be realized by means of a register, for example. The detection memory unit 178 stores various set values which are used for control by the detection control unit 179, such as the operation period for pulsed infrared radiation emission, intermittent operation time, insertion threshold, withdrawal threshold and LD current value.

The control unit 116 and the detection control unit 179 communicate. The control unit 116 and the detection control unit 179 communicate by means of a serial communication interface such as I2C (inter-integrated circuit) communication, for example. The control unit 116 controls operation of components of the light sensor unit 170 via the detection control unit 179.

For example, the control unit 116 performs control to switch a mode of the light sensor unit 170 to an operating mode for detecting reflected light or a sleep mode for stopping detection of reflected light. Specifically, in the sleep mode, the control unit 116 may perform control to stop the light-emitting unit 176 from emitting light, or may perform control to stop the light-receiving unit 177 from detecting reflected light. Furthermore, in the operating mode, the control unit 116 controls the light-emitting unit 176 to emit light and controls the light-receiving unit 177 to detect reflected light. By controlling switching of the mode of the light sensor unit 170 by means of the control unit 116, it is possible to reduce power consumption as compared to when reflected light is constantly being detected by means of the light sensor unit 170.

Furthermore, the control unit 116 causes the detection memory unit 178 to store the various set values which are used for control by the detection control unit 179. Furthermore, the control unit 116 receives various types of information such as interrupt notifications from the detection control unit 179, and reads out the information stored in the detection control unit 178.

Here, the detection memory unit 178 may be configured by a volatile storage medium or may be configured by a non-volatile storage medium. When the detection memory unit 178 is configured by a non-volatile storage medium, the various set values stored in the detection memory unit 178 are initialized when power supply to the light sensor unit 170 is interrupted and then power is once again supplied. When the various set values have been initialized, the control unit 116 may once again cause the detection memory unit 178 to store the various set values from before initialization.

It should be noted that, instead of the sleep mode, the control unit 116 may control the light sensor unit 170 to a power-off mode for stopping electrical supply to the light sensor unit 170. If control is performed in this way when the detection memory unit 170 is configured by a volatile storage medium, the control unit 116 causes the detection memory unit 178 to once again store the various set values from before initialization when the mode of the light sensor unit 170 is switched from the power-off mode to the operating mode. Furthermore, in the sleep mode, the control unit 116 may perform control to maintain electrical supply to the detection memory unit 178 provided in the light sensor unit 170. By this means, when the detection memory unit 178 is configured by a volatile storage medium, it is no longer necessary to cause the detection memory unit 178 to once again store the various set values from before initialization each time there is a switch from the sleep mode to the operating mode. Furthermore, in the sleep mode, the control unit 116 may perform control to maintain electrical supply only to a portion of the memory of the detection memory unit 178 provided in the light sensor unit 170. In the present description, the sleep mode and the power-off mode may also be referred to as the stop mode, as a general term for a mode in which detection is stopped.

The insertion status managed by the light sensor unit 170 when the light sensor unit 170 returns to the operating mode from the sleep mode need not continue the insertion status from before switching to the sleep mode, and may always be managed as “article not inserted”. In addition, an exception to the condition for sending an interrupt notification may be provided for when the light sensor unit 170 returns to the operating mode from the sleep mode. For example, a detection interrupt notification is sent when a detection value exceeding the insertion threshold is detected for the first time after a detection value falling below the withdrawal threshold was calculated by means of the detection control unit 179, as described above. As this exception, after the light sensor unit 170 has returned to the operating mode from the sleep mode, a detection interrupt notification may be sent when a detection value exceeding the insertion threshold has been detected, even if a detection value falling below the withdrawal threshold has not been detected. Similarly, after the light sensor unit 170 has returned to the operating mode from the sleep mode, a detection-deactivation interrupt notification may be sent when a detection value falling below the withdrawal threshold has been detected, even if a detection value exceeding the insertion threshold has not been detected by means of the detection control unit 179.

When the mode of one of the light sensor unit 170A and the light sensor unit 170B is the operating mode, the control unit 116 may set the mode of the other as the stop mode. This configuration makes it possible to prevent the occurrence of crosstalk. Crosstalk is a phenomenon by which light emitted from one of the light sensor unit 170A and the light sensor unit 170B is erroneously detected by the other.

### (3) Determination of Inserted Article

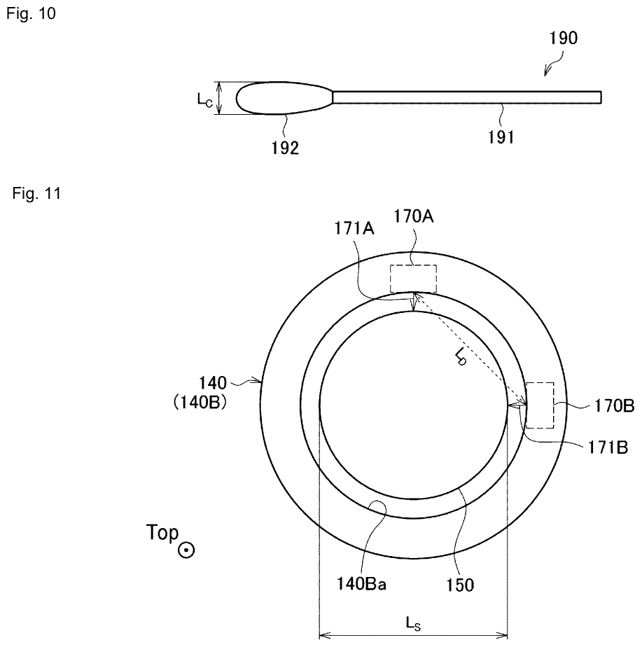

Adhered material such as soiling or a foreign object may remain in the internal space 141. As an example, contents may spill out from the tip end of the stick-type substrate 150 after heating, and may remain in the internal space 141 as adhered material. While adhered material remains, it is difficult to suitably heat the stick-type substrate 150, and as a result it is difficult for a good flavor to be provided to the user. The accommodating portion 140 is therefore preferably cleaned periodically. The adhered material is removed by means of cleaning, whereby it is possible to suitably heat the stick-type substrate 150, and as a result it is possible for a good flavor to be provided to the user. An example of a cleaning article used for cleaning the accommodating portion 140 will be described with reference to FIG. 10.

FIG. 10 shows an example of a configuration of a cleaning article 190 according to the embodiment. As shown in FIG. 10, the cleaning article 190 comprises a shaft portion 191 and a cleaning portion 192.

The shaft portion 191 is a member formed in a long shape. For example, the shaft portion 191 is formed by rolling a paper sheet.

The cleaning portion 192 may be formed by wrapping fibers onto one end of the shaft portion 191, and bonding the fibers thereto. The cleaning portion 192 may employ any shape, such as a teardrop shape, a cylindrical shape, a spherical shape, a shape having random unevenness, or a brush shape. Examples of fibers constituting the cleaning portion 192 which may be cited include various types of natural fibers (such as cotton, silk or wool), regenerated fibers (such as rayon or cupra), or synthetic fibers (such as polyester fibers or polypropylene fibers), etc. The cleaning portion 192 may contain a liquid such as an alcohol. It should be noted that the cleaning portion 192 may be disposed at one end of the shaft portion 191 as shown in FIG. 10, or may be disposed at both ends of the shaft portion 191.

The cleaning article 190 may be a cotton swab, for example. The user grips the shaft portion 191 and inserts the cleaning portion 192 into the internal space 141 from the opening 142. The user then moves the cleaning portion 192 while rubbing it against the accommodating portion 140. When this is done, the adhered material remaining in the accommodating portion 140 adheres to the cleaning portion 192 and is removed. The accommodating portion 140 is cleaned in this way.

The cleaning article 190 is formed to be narrower than the stick-type substrate 150. In particular, the diameter LC of the cleaning article 190 (more specifically, the diameter of the cleaning portion 192 constituting the thickest part) is formed to be shorter than the diameter LS of the stick-type substrate 150 (more specifically, the diameter of the narrowest part). As an example, the diameter LC of the cleaning article 190 may be no greater than half of the diameter LS of the stick-type substrate 150, and may preferably be no greater than one quarter thereof. This configuration ensures a large gap between the cleaning article 190 and the inner wall 140Ba of the guide portion 140B when the cleaning article 190 is inserted into the accommodating portion 140. As a result, the cleaning portion 192 can be freely moved in the internal space 141 and cleaning efficiency can be improved.

The cleaning article 190 is an example of an article other than the stick-type substrate 150 which could feasibly be inserted into the accommodating portion 140. Moreover, the inhalation device 100 and the cleaning article 190 may also be considered to constitute an aerosol-generating system. Alternatively, the inhalation device 100 may be understood to comprise the cleaning article 190.

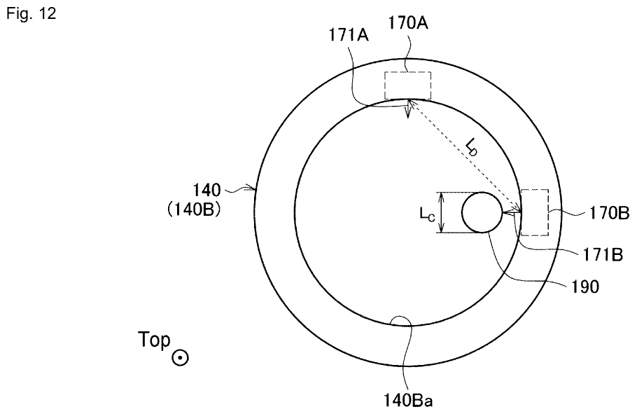

The difference between the diameter LC of the cleaning article 190 and the diameter LS of the stick-type substrate 150 may also be utilized to identify an article which has been inserted into the accommodating portion 140 (this will also be referred to below as an inserted article). This is because there is a large difference in detection values detected by means of the light sensor unit 170A and the light sensor unit 170B when the inserted article is the stick-type substrate 150 and when it is the cleaning article 190. This point will be explained with reference to FIGS. 11 and 12.

FIG. 11 schematically shows a situation in which the accommodating portion 140 having the stick-type substrate 150 inserted therein is seen from the opening 142 side (i.e., from the top). As shown in FIG. 11, the diameter LS of the stick-type substrate 150 is longer than the distance LD between the light sensor unit 170A and the light sensor unit 170B. As described with reference to FIG. 5 in regard to the clearance 175, the distance between the stick-type substrate 150 and the inner wall 140Ba of the guide portion 140B is around 1-2 mm. When the stick-type substrate 150 is inserted into the accommodating portion 140, as shown in FIG. 11, every part of the inner wall 140Ba of the guide portion 140B is therefore positioned in close proximity to the stick-type substrate 150. As a result, the light emitted by both the light sensor unit 170A and the light sensor unit 170B will be reflected by the stick-type substrate 150 positioned in close proximity. Accordingly, the detection value detected by means of the light sensor unit 170A and the detection value detected by means of the light sensor unit 170B will be largely equal values.

FIG. 12 schematically shows a situation in which the accommodating portion 140 having the cleaning article 190 inserted therein is seen from the opening 142 side (i.e., from the top). As shown in FIG. 12, the diameter LC of the cleaning article 190 is far shorter than the distance LD between the light sensor unit 170A and the light sensor unit 170B. When the cleaning article 190 is inserted into the accommodating portion, as shown in FIG. 12, the distance between the inner wall 140Ba of the guide portion 140B and the cleaning article 190 therefore greatly differs depending on the position on the inner wall 140Ba. As a result, the detection value of at least one of the light sensor unit 170A and the light sensor unit 170B is far smaller than when the stick-type substrate 150 is inserted into the accommodating portion 140. This is because the position of at least one of the light sensor unit 170A and the light sensor unit 170B is remote from the cleaning article 190, or is a position at which emitted light is not reflected by the cleaning article 190. In the example shown in FIG. 12, the detection value of the light sensor unit 170B has a similar magnitude to when the stick-type substrate 150 is inserted, but the detection value of the light sensor unit 170A is far smaller.

Here, the light sensor unit 170A and the light sensor unit 170B are arranged at the same position in the vertical direction, i.e., on the same circumference. By arranging the light sensor units 170 in this way, it is possible to perform detection by means of a plurality of light sensor units 170 even if the vertical length of the guide portion 140B is designed to be so small that a plurality of light sensor units 170 cannot be arranged at different positions in the vertical direction. That is to say, this configuration makes it possible to achieve a reduction in size of the inhalation device 100 because the vertical length of the guide portion 140B can be reduced. However, the light sensor unit 170A and the light sensor unit 170B are not limited to an arrangement in the same position in the vertical direction, and may equally be arranged at different positions in the vertical direction.

It should be noted that when the light sensor unit 170A and the light sensor unit 170B are arranged at the same position in the vertical direction, there is a greater possibility of light emitted from one of the light sensor units 170 being erroneously detected by the other light sensor unit 170. For this reason, only one of the light sensor unit 170A and the light sensor unit 170B is preferably in the operating mode. This makes it possible to prevent the occurrence of crosstalk.

The control unit 116 according to the embodiment therefore determines whether or not the inserted article is the stick-type substrate 150, based on detection values detected by means of the light sensor unit 170A and the light sensor unit 170B. More specifically, the control unit 116 determines whether or not the inserted article is the stick-type substrate 150, based on an interrupt notification sent in accordance with the detection values detected by means of the light sensor unit 170A and the light sensor unit 170B. As an example, the control unit 116 determines that the inserted article is the stick-type substrate 150 when a stick determination condition is satisfied.

The stick determination condition may be, for example, that a detection interrupt notification is received by either one of the light sensor unit 170A and the light sensor unit 170B within a predetermined time from reception of a detection interrupt notification by the other light sensor unit 170. The insertion threshold and withdrawal threshold used when an interrupt notification is sent should be freely set as values at which the stick determination condition is satisfied when the inserted article is the stick-type substrate 150, and at which the stick determination condition is not satisfied by at least either one of the light sensor unit 170A and the light sensor unit 170B when the inserted article is the cleaning article 190. However, the diameter of the stick-type substrate 150 varies according to brand or manufacturing lot, and it may also have an irregular shape. The insertion threshold is therefore preferably set at a value having a margin (i.e., a value on the low side). The insertion threshold is an example of a first threshold. A description will now mainly be given of an example in which the control unit 116 determines whether or not a detection interrupt notification is received by the light sensor unit 170B within a predetermined time from reception of a detection interrupt notification by the light sensor unit 170A.

As described above, the detection value of at least one of the light sensor unit 170A and the light sensor unit 170B when the cleaning article 190 is inserted into the accommodating portion 140 tends to be far smaller than when the stick-type substrate 150 is inserted into the accommodating portion 140. That is to say, when the cleaning article 190 is inserted into the accommodating portion 140, a detection interrupt notification is often not sent by the light sensor unit 170B immediately after a detection interrupt notification has been sent by the light sensor unit 170A (within a predetermined time). This configuration therefore makes it possible to prevent the cleaning article 190 from being erroneously determined as the stick-type substrate 150.

Meanwhile, the control unit 116 determines that the inserted article is not the stick-type substrate 150 when the stick determination condition is not satisfied. That is to say, the control unit 116 determines that the inserted article is not the stick-type substrate 150 when a detection interrupt notification is not received from the light sensor unit 170B within a predetermined time from reception of a detection interrupt notification from the light sensor unit 170A. The control unit 116 may determine that the inserted article is the cleaning article 190 when the stick determination condition is not satisfied.

The determination of whether or not the stick determination condition is satisfied may be made here by the control unit 116 comparing a detection value read from the light sensor unit 170 with the insertion threshold and the withdrawal threshold. That is to say, the control unit 116 may determine whether or not the stick determination condition is satisfied by reading a detection value from the light sensor unit 170 at any timing, without receiving an interrupt notification from the light sensor unit 170. In this case, for example, the stick determination condition may be that a detection value equal to or greater than the insertion threshold is also obtained by the light sensor unit 170B within a predetermined time from a detection value equal to or greater than the insertion threshold being obtained by the light sensor unit 170A.

An example of the stick determination condition has been described up to here. If the detection value during operation of one of the light sensor unit 170A and the light sensor unit 170B satisfies a predetermined condition (also referred to as a first condition) in the determination of whether or not the stick determination condition is satisfied, then the control unit 116 interrupts the stop mode of the other light sensor and switches it to the operating mode. The control unit 116 also interrupts the operating mode of the light sensor unit 170A or the light sensor unit 170B which detected the detection value satisfying the first condition, and switches that light sensor unit to the stop mode. The first condition is a partial condition of the stick determination condition, for example. Here, the stick determination condition is assumed to be that a detection interrupt notification is also received by either one of the light sensor unit 170A and the light sensor unit 170B within a predetermined time from reception of a detection interrupt notification by the other light sensor unit 170. In this case, the first condition may be that a detection interrupt notification is received by either one of the light sensor unit 170A and the light sensor unit 170B. That is to say, it can be said that the first condition in this case is that a detection value equal to or greater than the insertion threshold is detected by either one of the light sensor unit 170A and the light sensor unit 170B.

By controlling the light sensor units 170 so that only one of the light sensor unit 170A and the light sensor unit 170B is in the operating mode, it is thus possible to determine the inserted article based on the stick determination condition while preventing the occurrence of crosstalk. Furthermore, power consumption can be reduced as compared to when the light sensor unit 170A and the light sensor unit 170B are both in the operating mode.

The control unit 116 may determine whether the stick determination condition is satisfied by performing substitution control for switching the modes of the light sensor unit 170A and the light sensor unit 170B multiple times, so that the modes of the light sensor unit 170A and the light sensor unit 170B are substituted. In the substitution control, for example, the control unit 116 performs control to switch the light sensor unit 170A which is in the operating mode to the stop mode, and to switch the light sensor unit 170B which is in the stop mode to the operating mode. The substitution control may be performed each time a detection interrupt notification is received from the light sensor unit 170. Furthermore, the substitution control may be performed when a detection interrupt notification is not received within a predetermined time from the light sensor unit 170.

The stick determination condition when substitution control is performed multiple times may be that detection interrupt notifications are received from both light sensor units 170 a predetermined consecutive number of times, for example. If the condition is based on a detection interrupt notification being received once each from the light sensor unit 170A and the light sensor unit 170B, it is also conceivable that when the user moves the cleaning article 190 in the accommodating portion 140, both light sensor units 170 will send a detection interrupt notification according to the timing of detection. It is therefore possible to more reliably prevent the cleaning article 190 from being erroneously determined as the stick-type substrate 150 by basing the condition on a detection interrupt notification being received from both light sensor units 170 multiple times consecutively.

When substitution control is performed multiple times, if the stick determination condition includes a condition relating to the interrupt notification, an exception to the condition for sending an interrupt notification may be provided for when the sensor units 170 return to the operating mode from the sleep mode. More specifically, after the light sensor unit 170 has returned to the operating mode from the sleep mode, a detection interrupt notification is sent when a detection value exceeding the insertion threshold has been detected, even if a detection value falling below the withdrawal threshold has not been detected. By providing an exception in this way, if the stick-type substrate 150 is continuously inserted before and after substitution control is performed, the control unit 116 also receives a detection interrupt notification after substitution control has been performed. When substitution control is performed multiple times, the control unit 116 can therefore also determine insertion of the stick-type substrate 150 according to whether or not there are detection interrupt notifications.

Furthermore, when substitution control is performed multiple times, the stick determination condition may be determined by the control unit 116 reading out detection values from the light sensor units 170 each time substitution control is performed after a detection interrupt notification has been received once. For example, the control unit 116 may determine that the stick determination condition has been satisfied when a detection interrupt notification is received once, after which substitution control is performed, and the detection values from the light sensor units 170 read out after the substitution control are equal to or greater than the insertion threshold a predetermined consecutive number of times.

When substitution control is performed multiple times, the stick determination condition may include reception of a detection interrupt notification from the light sensor units 170 within a predetermined time from the substitution control being performed. The predetermined time when a detection interrupt notification has been received at least once from each of the two light sensor units 170 may be set shorter than the predetermined time when it is determined whether or not a detection interrupt notification has been received after substitution control has been performed for the first time. When substitution control is performed for the first time, it may be the case that the stick-type substrate 150 is in the process of being inserted into the accommodating portion 140. If a short predetermined time is set in this case, it is also conceivable that a detection value equal to or greater than a stick determination threshold will not be obtained by one of the light sensor units 170, depending on the orientation of insertion of the stick-type substrate 150 or the detection timing. However, it may be considered that the stick-type substrate 150 has been fully inserted into the accommodating portion 140 when a detection interrupt notification has been received at least once from each of the two light sensor units 170. It is therefore possible to determine more quickly whether or not the stick determination condition has been satisfied by setting the predetermined time in this case to be shorter than the predetermined time when it is determined whether or not a detection interrupt notification has been received after substitution control has been performed for the first time.

Furthermore, the stick determination condition when substitution control is performed multiple times may be a condition based on detection results detected by the light sensor unit 170A and the light sensor unit 170B by performing substitution control a predetermined number of times, for example. For example, the stick determination condition may be that substitution control is performed a first predetermined number of times (e.g., 10 times), and the cumulative total of detection interrupt notifications received from the light sensor unit 170A and the light sensor unit 170B is equal to or greater than a second predetermined number of times (e.g., 8 times). As a different example, the stick determination condition may be that substitution control is performed the first predetermined number of times (e.g., 10 times), and detection interrupt notifications are received every time from the light sensor unit 170A or the light sensor unit 170B after substitution control has been performed a third predetermined number of times (e.g., the last five times), counting from the last substitution control.

It should be noted that when the determination of whether or not the stick determination condition is satisfied is made by the control unit 116 comparing a detection value read from the light sensor unit 170 with the insertion threshold and the withdrawal threshold, the stick determination condition may also include a condition relating to the number of times of detection performed by the light sensor 170, instead of time. For example, the stick determination condition may include a detection value equal to or greater than the insertion threshold being obtained by the light sensor unit 170 from detection within a predetermined number of times from substitution control being performed. Here, when a detection value equal to or greater than the insertion threshold is detected at least once each by the two light sensor units 170, the stick determination condition may also include a detection value equal to or greater than the insertion threshold being detected by detection by the light sensor units 170 performed immediately after substitution control was performed.

Meanwhile, the control unit 116 determines that the inserted article is not the stick-type substrate 150 when a detection interrupt notification is not received from the light sensor units 170 within a predetermined time from substitution control being performed, that is, when a detection interrupt notification is not received a predetermined consecutive number of times.

The control unit 116 according to the embodiment further determines whether or not the inserted stick-type substrate 150 has been withdrawn, based on the detection values detected by means of the light sensor units 170. As an example, after the stick determination condition has been satisfied, the control unit 116 determines that the stick-type substrate 150 has been withdrawn when a stick withdrawal determination condition (also referred to as a second condition) has been satisfied. The stick withdrawal determination condition may be, for example, that a detection-deactivation interrupt notification has been received from either one of the light sensor unit 170A and the light sensor unit 170B. That is to say, it can be said that the stick withdrawal determination condition in this case is also that a detection value equal to or less than the withdrawal threshold is obtained by either one of the light sensor unit 170A and the light sensor unit 170B.

Furthermore, the control unit 116 may determine whether or not the stick withdrawal determination condition is satisfied by performing substitution control multiple times. The stick withdrawal determination condition when substitution control is performed multiple times may be that detection-deactivation interrupt notifications are received from both light sensor units 170 a predetermined consecutive number of times, for example.

As a more specific example, the control unit 116 first of all receives a detection-deactivation interrupt notification from one of the light sensor units 170, and then performs substitution control. If a detection-deactivation interrupt notification is also received from the other light sensor unit 170 after the substitution control, the control unit 116 may then determine that the stick withdrawal determination condition has been satisfied. Meanwhile, if a detection-deactivation interrupt notification is not received from the other light sensor unit 170 after substitution control has been performed, the control unit may determine that the stick withdrawal determination condition is not satisfied. That is to say, the control unit 116 may determine in this case that the stick-type substrate 150 is still inserted.

Depending on the situation in which the inhalation device 100 is placed, it is also conceivable that the light sensor unit 170 will send a detection-deactivation interrupt notification because of detection values fluctuating up and down due to the effects of external interference, etc., despite the fact that the stick-type substrate 150 has not been withdrawn. In such a case, it is also conceivable that withdrawal of the stick-type substrate 150 will be erroneously determined if a determination of the stick withdrawal determination condition is made because of a detection-deactivation interrupt notification being received from either one of the light sensor unit 170A and the light sensor unit 170B. It is therefore possible to prevent such erroneous determinations by determining the stick withdrawal determination condition on the basis of detection values obtained by performing substitution control multiple times, improving the accuracy of determining withdrawal of the stick-type substrate 150.

When substitution control is performed multiple times, if the stick withdrawal determination condition includes a condition relating to the interrupt notification, an exception to the condition for sending an interrupt notification may be provided for when the sensor units 170 return to the operating mode from the sleep mode. Furthermore, when substitution control is performed multiple times, the stick withdrawal determination condition may be determined by the control unit 116 reading out detection values from the light sensor units 170 each time substitution control is performed after a detection-deactivation interrupt notification has been received once.

Moreover, the control unit 116 may determine whether the stick determination condition and the stick withdrawal determination condition have been satisfied by reading out the insertion status stored in the detection memory unit 178, following reception of an interrupt notification sent without distinguishing between a detection interrupt notification and a detection-deactivation interrupt notification. For example, when an interrupt notification is sent from the light sensor unit 170 and the insertion status is read as “article not inserted”, the control unit 116 may determine that the stick withdrawal determination condition has been satisfied.

A summary will be given here of switching of the mode of the light sensor units 170 by the control unit 116 for an inserted article determination and an inserted article withdrawal determination. As an example, the control unit 116 performs control so that the light sensor unit 170A is in the operating mode and the light sensor unit 170B is in the stop mode, and then stands by for article insertion. When a detection interrupt notification is received from the light sensor unit 170A, the control unit 116 then interrupts the operating mode of the light sensor unit 170A and switches it to the stop mode. The control unit 116 also interrupts the stop mode of the light sensor unit 170B and switches it to the operating mode. Here, if a detection interrupt notification is received from the light sensor unit 170B within a predetermined time, the control unit 116 determines that the inserted article is the stick-type substrate 150 and maintains the state of detection being performed by only the light sensor unit 170B, without switching the modes of either of the light sensor units 170.

If a detection-deactivation interrupt notification is received from the light sensor unit 170B, the control unit 116 may then determine that the stick-type substrate 150 has been withdrawn, and may switch the mode of the light sensor unit 170B from the operating mode to the stop mode. The control unit 116 may also perform control to switch the mode of the light sensor unit 170A to the operating mode. Furthermore, if it is determined that the inserted article is the cleaning article 190, the control unit 116 may likewise perform control to switch the mode of the light sensor unit 170B which is in the operating mode to the stop mode, and to switch the mode of the light sensor unit 170A which is in the stop mode to the operating mode. By performing control in this way, the light sensor unit 170 operating during standby for insertion of an article and the light sensor unit 170 operating during standby for withdrawal of the stick-type substrate 150 will always be the same light sensor unit 170. Here, the light sensor unit 170A is always in the operating mode during standby for insertion of an article. Furthermore, the light sensor unit 170B is always in the operating mode during standby for withdrawal of the stick-type substrate 150. By controlling the modes of each of the light sensor units 170 in this way, it is possible to simplify control of each of the light sensor units 170 because each light sensor unit 170 has a limited role.

### (4) Control of Heating Correspondingly with Inserted Article Determination Result

The control unit 116 may control operation of the heating unit 121 on the basis of a detection value obtained by the light sensor unit 170A or the light sensor unit 170B. For example, the control unit 116 may control operation of the heating unit 121 on the basis of the result of determining whether or not the inserted article is the stick-type substrate 150. More specifically, the control unit 116 varies the operation of the heating unit 121 when the inserted article is the stick-type substrate 150 and when this is not the case. This configuration makes it possible to further improve usability.

As an example, the control unit 116 may start heating by the heating unit 121 when it has been determined that the inserted article is the stick-type substrate 150. This determination result may be achieved according to whether or not the stick determination condition, including the first condition, has been satisfied, for example. The heating afforded by the heating unit 121 which is started in accordance with the inserted article determination result will also be referred to here as automatic heating. On the other hand, the control unit 116 does not cause automatic heating by the heating unit 121 when it is determined that the inserted article is not the stick-type substrate 150. That is to say, the control unit 116 may perform automatic heating only when the stick-type substrate 150 is inserted. This configuration makes it possible to improve usability because automatic heating is performed simply by insertion of the stick-type substrate 150 into the accommodating portion 140, even if no separate user operation is performed to instruct the start of heating, such as pressing of a button.

As another example, the control unit 116 may stop heating by the heating unit 121 based on the result of determining whether or not the inserted stick-type substrate 150 has been withdrawn. For example, during heating by the heating unit 121, the control unit 116 controls the mode of either one of the light sensor unit 170A and the light sensor unit 170B to the operating mode, and controls the mode of the other to the stop mode. If the detection value detected by means of the light sensor unit 170A or the light sensor unit 170B satisfies the stick withdrawal determination condition during heating by the heating unit 121, the control unit 116 then performs control to stop heating by the heating unit 121. Higher accuracy in determining withdrawal of the stick-type substrate 150 by the control unit 116 makes it possible to better prevent automatic heating from being stopped at a timing contrary to the user's expectation.

As another example, the control unit 116 may permit heating by the heating unit 121 when it has been determined that the inserted article is the stick-type substrate 150, and may prohibit heating by the heating unit 121 when it has been determined that the inserted article is not the stick-type substrate 150. If heating is permitted, the inhalation device 100 starts heating when a user operation to instruct the start of heating, such as pressing of a button, has been performed. If heating is prohibited, on the other hand, the inhalation device 100 does not start heating even if a user operation to instruct the start of heating, such as pressing of a button, has been performed. This configuration makes it possible to improve user safety because heating is not started even if the button is erroneously operated during cleaning.

### (5) Control Processing Flow for Inserted Article Determination and Automatic Heating

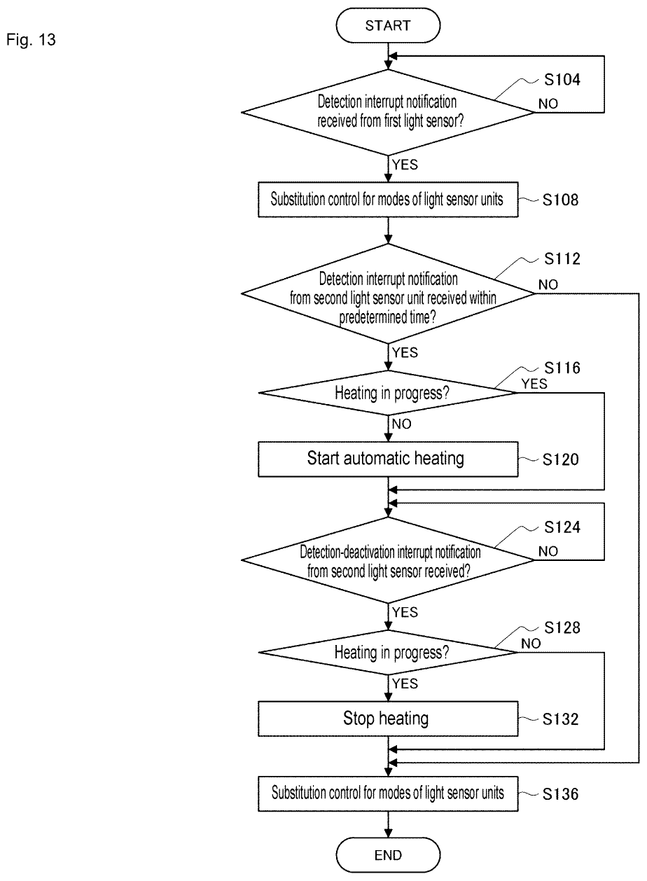

Control processing for automatic heating executed by the inhalation device 100 according to the embodiment will be described next with the aid of FIG. 13. FIG. 13 is a flowchart showing an example of a control processing flow for automatic heating executed by the inhalation device 100 according to the embodiment.

As shown in FIG. 13, the control unit 116 first of all determines whether or not a detection interrupt notification from the light sensor unit 170A (first light sensor) has been received (S104). Until a detection interrupt notification is received from the light sensor unit 170A, the control unit 116 continues to control the light sensor units 170 so that the mode of the light sensor unit 170A is the operating mode and so that the mode of the light sensor unit 170B (second light sensor) is the stop mode (S104/NO). When a detection interrupt notification has been received from the light sensor unit 170A (S104/YES), the control unit 116 performs substitution control with respect to the modes of the light sensor units 170 (S108). That is to say, the control unit 116 performs control to switch the mode of the light sensor unit 170A to the stop mode, and to switch the mode of the light sensor unit 170B to the operating mode.

The control unit 116 then determines whether or not a detection interrupt notification from the light sensor unit 170B has been received within a predetermined time (S112). If the control unit 116 has received a detection interrupt notification from the light sensor unit 170B within the predetermined time (S112/YES), the control unit determines that the inserted article is the stick-type substrate 150 and the processing advances to S116. On the other hand, if the control unit 116 has not received a detection interrupt notification from the light sensor unit 170B within the predetermined time (S112/NO), the control unit 116 determines that the inserted article is not the stick-type substrate 150 and the processing advances to S136.

If it has been determined that the inserted article is the stick-type substrate 150, the control unit 116 determines whether or not heating by the heating unit 121 is being implemented (S116). If heating is being implemented by the heating unit 121 (S116/YES), the control unit 116 advances the processing to S124. If heating is not being implemented by the heating unit 121 (S116/NO), the control unit 116 starts automatic heating by the heating unit 121 (S120).

The control unit 116 then determines whether or not a detection-deactivation interrupt notification from the light sensor unit 170B has been received (S124). If a detection-deactivation interrupt notification from the light sensor unit 170B has been received, the control unit 116 determines that the stick-type substrate 150 has been withdrawn and the processing advances to S128 (S124/YES). The control unit 116 continues to control the mode of the light sensor unit 170B to the operating mode until a detection-deactivation interrupt notification from the light sensor unit 170B is received (S124/NO).

If it has been determined that the stick-type substrate 150 has been withdrawn, the control unit 116 determines whether or not heating by the heating unit 121 is being implemented (S128). If heating is not being implemented by the heating unit 121 (S128/NO), the control unit 116 advances the processing to S136. If heating is being implemented by the heating unit 121 (S128/YES), the control unit 116 stops heating by the heating unit 121 (S132). The control unit 116 then performs substitution control with respect to the modes of the light sensor units 170 and terminates the processing (S136). That is to say, the control unit 116 performs control to switch the mode of the light sensor unit 170A to the operating mode, and to switch the mode of the light sensor unit 170B to the stop mode. The flow described up to this point with the aid of FIG. 13, in which automatic heating is performed and heating is stopped after an inserted article determination has been made in accordance with detection values detected by means of the light sensor units 170, will be referred to as the automatic heating control flow.

### (6) Flow of Inserted Article Determination Processing Based on Multiple Substitution Control Operations

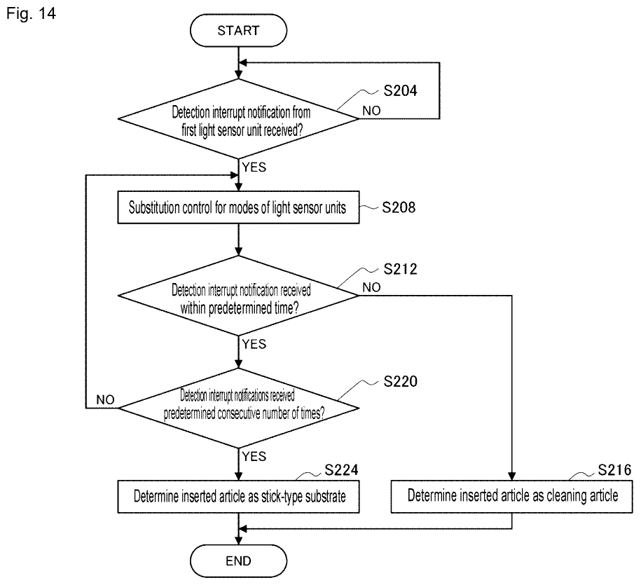

Inserted article determination processing based on multiple substitution control operations, which is executed by the inhalation device 100 according to the embodiment, will be described next with the aid of FIG. 14. This determination processing may be applied in place of S104-S112 in the automatic heating control flow described with the aid of FIG. 13. When the processing is applied in this way, the processing advances to S136 in FIG. 13 after S216 in FIG. 14. Furthermore, the processing advances to S116 in FIG. 13 after S224 in FIG. 14.

FIG. 14 is a flowchart illustrating an example of a determination processing flow for an inserted article based on multiple substitution control operations, which is executed by the inhalation device 100 according to the embodiment. The control unit 116 first of all determines whether or not a detection interrupt notification from the light sensor unit 170A has been received (S204). Until a detection interrupt notification is received from the light sensor unit 170A, the control unit 116 continues to control the light sensor units 170 so that the mode of the light sensor unit 170A is the operating mode and so that the mode of the light sensor unit 170B is the stop mode (S204/NO). When a detection interrupt notification has been received from the light sensor unit 170A (S204/YES), the control unit 116 performs substitution control with respect to the modes of the light sensor units 170 (S208). That is to say, the control unit 116 performs control to switch the mode of the light sensor unit 170A to the stop mode, and to switch the mode of the light sensor unit 170B to the operating mode.

The control unit 116 then determines whether or not a detection interrupt notification from the light sensor unit 170 has been received within a predetermined time (S212). If the control unit 116 has not received a detection interrupt notification from the light sensor unit 170 within the predetermined time (S212/NO), the control unit 116 determines that the inserted article is the cleaning article 190 and terminates the processing (S216). Here, if the mode of the light sensor unit 170A is the stop mode and the mode of the light sensor unit 170B is the operating mode, the control unit 116 may perform substitution control so that the mode of the light sensor unit 170A becomes the operating mode and the mode of the light sensor unit 170B becomes the stop mode. The mode of each of the light sensor units 170 is restored to the initial state by this means.

Meanwhile, if the control unit 116 has received a detection interrupt notification from the light sensor unit 170 within the predetermined time (S212/YES), the control unit 116 determines whether detection interrupt notifications have been received a predetermined consecutive number of times (S220). If detection interrupt notifications have not been received a predetermined consecutive number of times (S220/NO), the control unit 116 repeats the processing of S208-S212. If detection interrupt notifications have been received a predetermined consecutive number of times (S220/YES), the control unit 116 determines that the inserted article is the stick-type substrate 150 and terminates the processing (S224).

### (7) Control in Accordance with Detection Result from Sensor Unit 112

A description will be given next of control by the control unit 116 in accordance with a detection result obtained by the sensor unit 112. The sensor unit 112 is an example of a second detection unit of this embodiment, which detects information relating to the state of the inhalation device 100.

A user instruction relating to operation of the inhalation device 100 is an example of information relating to the state of the inhalation device 100 detected by the sensor unit 112. The sensor unit 112 is capable of detecting instructions to start and stop heating by the heating unit 121, for example. Furthermore, the sensor unit 112 is capable of detecting an instruction to start prohibiting the use of various functions and an instruction to cancel the prohibition. Furthermore, the sensor unit 112 is capable of detecting an instruction to shift to a state in which heating by the heating unit 121 is prohibited or an instruction to cancel the state in which heating is prohibited. Input of the instruction to shift to a state in which heating by the heating unit 121 is prohibited may be, for example, input of an instruction to shift to a locked state in which predetermined control associated with input, other than predetermined operational input, is not performed even if there is such input. The locked state is a state in which heating control is not performed even if a heating start instruction (input other than predetermined operational input) for the heating unit 121 is input. Even in the locked state, the inhalation device 100 accepts predetermined operational input such as input of an operation to cancel the locked state or setting of an operating pattern for shifting to the locked state, and implements the corresponding control, for example.

Furthermore, the sensor unit 112 is capable of detecting an instruction to start prohibiting detection by the light sensor units 170 and an instruction to cancel the detection prohibition. It should be noted that the instruction to start prohibiting detection by the light sensor units 170 and the instruction to cancel the detection prohibition may be accepted only when the opening 142 is closed by means of the cover portion 14. This configuration makes it possible to prevent automatic heating from being implemented unintentionally by the user when an instruction to cancel the prohibition on detection by the light sensor unit 170 has been given. Furthermore, if the light sensor units 170 are calibrated when the mode of the light sensor units 170 switches from the stop mode to the operating mode, it is possible to perform the calibration while eliminating deviations in detection values caused by the effects of external light.

Furthermore, the sensor unit 112 may detect an instruction to cause the inhalation device 100 to sleep, or to cancel sleeping. When the inhalation device 100 is caused to sleep, the inhalation device 100 stops some of the functions of the inhalation device 100, such as heating by the heating unit 121, until the sensor unit 112 detects an instruction to cancel sleeping. It should be noted that instructions relating to sleeping of the inhalation device 100 need not be input by the user, and may, for example, be input by means of the control unit 116 based on the time elapsed from the last operation of the inhalation device 100.

The instructions detected by the sensor unit 112 may also be detected by pressing of a button included in the sensor unit 112. The sensor unit 112 may detect instructions by the length of time for which the button is pressed or the number of times the button is pressed, etc. For example, the sensor unit 112 may detect a short press of the button as an instruction to cause the inhalation device 100 to sleep or to cancel sleeping. Furthermore, the sensor unit 112 may detect a long press of the button as an instruction to start or stop heating by the heating unit 121.

The sensor unit 112 may furthermore comprise a motion sensor. Detection of movement by the motion sensor may allow the sensor unit 112 to detect an instruction for a preset operation in accordance with the movement detected by the motion sensor. Furthermore, instructions detected by the sensor unit 112 may be received by means of the communication unit 115 from a communication terminal such as a smartphone used by the user.

A state of the inhalation device 100 in which an error has occurred is another example of information relating to the state of the inhalation device 100 detected by the sensor unit 112. As an example, the sensor unit 112 may detect the start and clearing of an automatic resolution-possible error state, which is a state where an error has occurred, and the error can be automatically resolved by means of control performed by the control unit 116. An automatic resolution-possible error is, for example, an error indicating that the temperature inside or outside the inhalation device 100, such as the temperature of the power source unit 111, has an abnormal value. In order to resolve a state in which such an error has occurred, the control unit 116 controls the heating unit 121 to stop heating or controls the power source unit 111 to stop charging, until this temperature reaches a normal temperature within a predetermined temperature range. When an automatic resolution-possible error has occurred, the control unit 116 is thus capable of automatically resolving the automatic resolution-possible error state without an accompanying user operation. As another example, the sensor unit 112 may detect the start and clearing of an automatic resolution-impossible error state, which cannot be automatically resolved by the control unit 116. An automatic resolution-impossible error may be an error which necessitates resetting of hardware in order to resolve the error, for example.

Connection and disconnection of charging of the power source unit 111 by the user is another example of information relating to the state of the inhalation device 100 detected by the sensor unit 112. Furthermore, the information relating to the state of the inhalation device 100 may be opening/closing of the opening 142 by the cover portion 14.

The start or termination of switching of a heating profile by means of the control unit 116 is another example of information relating to the state of the inhalation device 100 detected by the sensor unit 112. The heating profile indicates a time-series transition of heating performed by the heating unit 121. The heating unit 121 performs heating in accordance with the heating profile. The heating profile may be switched by a user operation of a button included in the sensor unit 112, or may be switched on the basis of the communication unit 115 receiving settings information from a communication terminal such as a smartphone used by the user.

Information relating to the state of the inhalation device 100 which is detected by means of the sensor unit 112 has been described thus far. The control unit 116 controls switching of the modes of the light sensor units 170 in accordance with detection results obtained by means of the sensor unit 112. Here, when there are multiple light sensor units 170, the control unit 116 controls mode switching for each of the plurality of light sensor units 170.

The control unit 116 also decides whether or not to control an operation of the inhalation device 100 in accordance with detection values detected by means of the light sensor units 170 when the mode of the light sensor units 170 is switched to the operating mode. Controlling an operation of the inhalation device 100 in accordance with detection values detected by means of the light sensor units 170 may be controlling heating in accordance with an inserted article determination result which was described above, for example. Furthermore, as another example, controlling an operation of the inhalation device 100 in accordance with detection values detected by means of the light sensor units 170 may also be controlling transmission of a notification prompting a user to clean the accommodating portion 140, which is notified in accordance with the detection values detected by means of the light sensor units 170. The description from here will mainly be an exemplary case in which controlling an operation of the inhalation device 100 in accordance with detection values detected by means of the light sensor unit 170 is controlling heating (automatic heating control) in accordance with an inserted article determination result.

The sensor unit 112 detecting opening/closing of the opening 142 by means of the cover portion 14 will be described first of all. It is very likely that the user will use the inhalation device 100 when the opening 142 has been opened by the cover portion 14. When the sensor unit 112 detects opening of the opening 142 by the cover portion 14, the control unit 116 therefore controls the mode of the light sensor unit 170 from the stop mode to the operating mode. Here, when there are multiple light sensor units 170, the control unit 116 may control mode switching so that the mode of one of the plurality of light sensor units 170 is the operating mode.

Meanwhile, the user will not be using the inhalation device 100 when the opening 142 has been closed by the cover portion 14. When the sensor unit 112 detects closure of the opening 142 by the cover portion 14, the control unit 116 therefore performs control to switch the mode of the light sensor unit 170 from the operating mode to the stop mode. Here, when there are multiple light sensor units 170, the control unit 116 may control mode switching so that the modes of all of the plurality of light sensor units 170 are the stop mode. This configuration makes it possible to effectively reduce power consumption because detection by the light sensor units 170 is performed only when the user is using the inhalation device 100.

When the sensor unit 112 detects an instruction to start prohibiting detection by the light sensor unit 170 and an instruction to cancel the detection prohibition, the control unit 116 controls switching of the mode of the light sensor unit 170 in accordance with the instruction. Specifically, when the sensor unit 112 detects an instruction to start prohibiting detection by the light sensor unit 170, the control unit 116 performs control to switch the mode of the light sensor unit 170 from the operating mode to the stop mode. Here, when there are multiple light sensor units 170, the control unit 116 may control mode switching so that the mode of one of the plurality of light sensor units 170 is the operating mode.

Furthermore, when the sensor unit 112 detects an instruction to cancel the prohibition of detection by the light sensor unit 170, the control unit 116 performs control to switch the mode of the light sensor unit 170 from the stop mode to the operating mode. Here, when there are multiple light sensor units 170, the control unit 116 may control mode switching so that the modes of all of the plurality of light sensor units 170 are the stop mode.

If the sensor unit 112 detects that the inhalation device 100 is in an automatic resolution-impossible error state when detection of an automatic resolution-impossible error state of the inhalation device 100 is performed, the control unit 116 performs control to switch the mode of the light sensor unit 170 from the operating mode to the stop mode. Here, when there are multiple light sensor units 170, the control unit 116 may control mode switching so that the mode of one of the plurality of light sensor units 170 is the operating mode.

Furthermore, when clearing of the automatic resolution-impossible error state of the inhalation device 100 is detected, the control unit 116 performs control to switch the mode of the light sensor unit 170 from the stop mode to the operating mode. Here, when there are multiple light sensor units 170, the control unit 116 may control mode switching so that the modes of all of the plurality of light sensor units 170 are the stop mode.

The control unit 116 decides that automatic heating control should be performed when the sensor unit 112 detects opening of the opening 142 by the cover portion 14, an instruction to start prohibiting detection by the light sensor unit 170, or clearing of an automatic resolution-impossible error state. For example, when the sensor unit 112 detects opening of the opening 142 by the cover portion 14, the control unit 116 controls automatic heating after the light sensor unit 170 has been switched to the operating mode.

A case in which the sensor unit 112 detects a first operation or a second operation will be described next. The first operation includes: connection of charging of the power source unit 111, input of an instruction to shift to a state in which heating by the heating unit 121 is prohibited, the start of switching of the heating profile by means of the control unit 116, the start of an automatic resolution-possible error state, or an instruction to cause the inhalation device 100 to sleep. Furthermore, the second operation includes: disconnection of charging of the power source unit 111, input of an instruction to shift to a state in which heating by the heating unit 121 can be implemented, termination of switching of the heating profile by means of the control unit 116, clearing of an automatic resolution-possible error state, or an instruction to cancel sleeping of the inhalation device 100.

When the first operation has been detected, the control unit 116 controls the light sensor unit 170 so that the mode of the light sensor unit 170 is the operating mode. It should be noted that when the light sensor unit 170 was in the operating mode before the first operation was detected, the operating mode may be maintained. Here, when there are multiple light sensor units 170, the control unit 116 may control the plurality of light sensor units 170 so that the mode of only one of the plurality of light sensor units 170 is the operating mode.

Furthermore, when the first operation has been detected by the sensor unit 112, the control unit 116 decides that automatic heating should not be controlled until a second operation corresponding to the detected first operation is detected. The period of time until the second operation corresponding to the detected first operation is detected will be referred to as the second operation standby period. For example, when connection of charging to the power source unit 111 has been detected, the control unit 116 decides that automatic heating control should not be performed until disconnection of charging from the power source unit 111 is detected. The control unit 116 performs control so that the state of the inhalation device 100 during the second operation standby period is an automatic heating prohibition state in which automatic heating control is not performed. This configuration makes it possible to improve safety or convenience for the user because it prevents automatic heating of the stick-type substrate 150 at a timing when inhalation by the user is not expected. Moreover, if heating by the heating unit 121 is being performed when the first operation is detected by means of the sensor unit 112, the control unit 116 controls the heating unit 121 to stop heating.

It will be assumed here that the first operation was detected by the sensor unit 112 during the time from after a determination that the inserted article is the stick-type substrate 150, until it is determined that the stick-type substrate 150 has been withdrawn. In this case, the control unit 116 continues the automatic heating prohibition state until it is determined that the stick-type substrate 150 has been withdrawn. That is to say, the control unit 116 decides that automatic heating control should not be performed until it is determined that the stick-type substrate 150 has been withdrawn. This makes it possible to prevent the stick-type substrate 150 which was inserted into the accommodating portion 140 before the first operation was detected from being suddenly heated after the second operation has been detected, contrary to the user's expectation.

It will furthermore be assumed that the inserted article was determined as the stick-type substrate 150 during the time from after detection of the first operation by the sensor unit 112 until detection of the second operation by the sensor unit 112, i.e., during the automatic heating prohibition state. In this case, the control unit 116 continues the automatic heating prohibition state until it is determined that the inserted article has been withdrawn. That is to say, the control unit 116 decides that automatic heating control should not be performed until it is determined that the inserted article has been withdrawn. This makes it possible to prevent the stick-type substrate 150 which was inserted during the automatic heating prohibition state from being suddenly heated after the second operation has been detected, contrary to the user's expectation.

In order to prevent heating contrary to the user's expectation, the control unit 116 may reset the automatic heating control flow when the first operation has been detected, or when it has been determined that the inserted article is the stick-type substrate 150 during the second operation standby period or that the stick-type substrate 150 has been withdrawn. That is to say, the control unit 116 may reset the automatic heating control flow when the first operation has been detected, or when an interrupt notification has been received during the second operation standby period. Resetting the automatic heating control flow comprises terminating the automatic heating control flow being processed, and restarting the automatic heating control flow. Moreover, if heating by the heating unit 121 is being performed when the automatic heating control flow is terminated, the control unit 116 performs controls to stop heating. Resetting the automatic heating control flow makes it possible to prevent heating contrary to the user's expectation when it was determined before the first operation is detected that the inserted article is the stick-type substrate 150, or when it was determined during the automatic heating prohibition state that the inserted article is the stick-type substrate 150.

### (8) Control Processing for Automatic Heating in Accordance with Detection Result of Sensor Unit 112

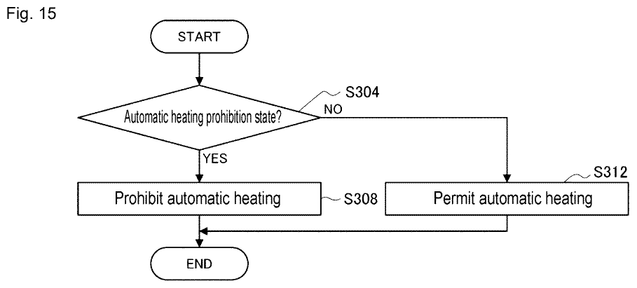

Control processing for automatic heating in accordance with a detection result of the sensor unit 112, which is executed by the inhalation device 100 according to the embodiment will be described next. FIG. 15 is a flowchart illustrating an example of a control processing flow for automatic heating in accordance with a detection result of the sensor unit 112, which is executed by the inhalation device 100 according to the embodiment.

As shown in FIG. 15, the control unit 116 first of all determines whether the state of the inhalation device 100, which is decided in accordance with the detection result of the sensor unit 112, is the automatic heating prohibition state (S304). If the state of the inhalation device 100 is the automatic heating prohibition state (S304/YES), the control unit 116 prohibits automatic heating by the heating unit 121 and terminates the processing (S308). Meanwhile, if the state of the inhalation device 100 is not the automatic heating prohibition state (S304/NO), the control unit 116 permits automatic heating by the heating unit 121 and terminates the processing (S312).

### (9) Control Processing for Resetting Automatic Heating Control Flow

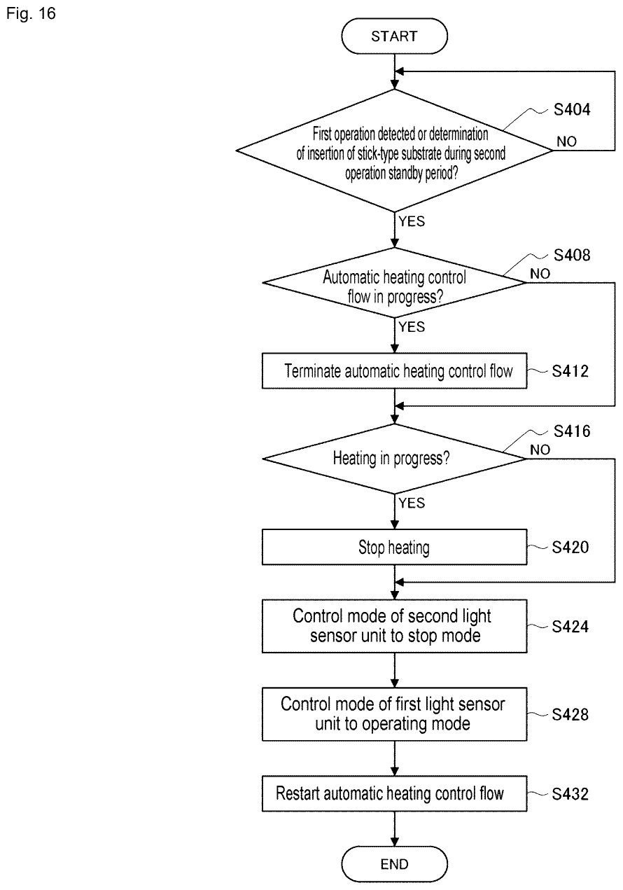

Control processing for resetting the automatic heating control flow, which is executed by the inhalation device 100 according to the embodiment will be described next. FIG. 16 is a flowchart showing an example of a control processing flow for resetting the automatic heating control flow, which is executed by the inhalation device 100 according to the embodiment.

As shown in FIG. 16, the control unit 116 first of all determines whether the first operation has been detected or whether an interrupt notification was received during the second operation standby period (S404). The control unit 116 continues to cause detection by the sensor unit 112 and the light sensor unit 170 until the first operation is detected or until an interrupt notification is received during the second operation standby period (S404/NO). Meanwhile, if the first operation is detected or an interrupt notification is received during the second operation standby period (S404/YES), the control unit 116 terminates the automatic heating control flow in progress (S412). The control unit 116 then determines whether heating by the heating unit 121 is in progress (S416).

If heating is not being implemented by the heating unit 121 (S416/NO), the control unit 116 advances the processing to S424. If heating is being implemented by the heating unit 121 (S416/YES), the heating unit 121 stops heating (S420). The control unit 116 then controls the light sensor unit 170B so that the mode of the light sensor unit 170B is the stop mode (S424). Furthermore, the control unit 116 controls the light sensor unit 170A so that the mode of the light sensor unit 170A is the operating mode (S428). The control unit 116 then restarts the automatic heating control flow (S432).

### 3. Supplement

Although preferred embodiments of the present disclosure have been described in detail above with reference to the accompanying drawings, the present disclosure is not limited to such examples. It is obvious that a person having an ordinary level of knowledge in the technical field to which the present disclosure belongs could conceive of various modified examples or variations within the scope of the technical concepts set forth in the claims, and these modified examples and variations will naturally be understood to fall within the technical scope of the present disclosure.

The embodiments above described an example in which the inhalation device 100 comprises two light sensor units 170, but the present disclosure is not limited to this example, and the inhalation device 100 may equally comprise three or more light sensor units 170. In this case, the control unit 116 controls only one of the three or more light sensor units 170 to the operating mode, and then stands by for article insertion. When a detection interrupt notification is received from the light sensor unit 170 which is in the operating mode, the control unit 116 performs control so that any one of the other light sensor units 170 is in the operating mode. The control unit 116 repeats this control until a detection interrupt notification is received from all of the light sensor units 170.

Furthermore, the embodiments above described an example in which the state of the internal space 141 is detected by the light sensor units 170, but examples of a state detection unit for detecting the state of the internal space 141 are not limited to this. For example, the internal space 141 may equally be detected by means of a capacitive sensor instead of the light sensor units 170.

It should be noted that the series of processes performed by each device described in the present description may be realized by using software, hardware, and any combination of software and hardware. Programs constituting the software are prestored on a recording medium (more specifically, a non-transitory computer-readable storage medium) provided internally or externally to each device, for example. When the programs are then executed, for example, by a computer for controlling each device described in the present description, the programs are read into a RAM and executed by means of a processing circuit such as a CPU. The recording medium is, for example, a magnetic disk, an optical disk, a magneto-optical disk, or a flash memory, etc. Furthermore, the computer programs may be distributed via a network, for example, without the use of a recording medium. Furthermore, the computer may be an application-specific integrated circuit such as ASIC, a general-purpose processor which executes functions by reading software programs, or a computer on a server used for cloud computing, etc. Furthermore, the series of processes performed by each device described in the present description may be processed in a distributed manner by multiple computers.

Furthermore, the processing described using flowcharts and sequence diagrams in the present description need not necessarily be implemented in the order depicted. Some processing steps may be implemented in parallel. Furthermore, additional processing steps may be employed and some processing steps may be omitted.

It should be noted that configurations such as the following also fall within the technical scope of the present disclosure.
(1)

An inhalation device comprising:
an accommodating portion having an internal space and an opening enabling the internal space to communicate with the outside;
a first detection unit for detecting a state of the internal space; and
a second detection unit for detecting information relating to a state of the inhalation device; and
a control unit for performing, based on a detection result obtained by means of the second detection unit: control to switch a mode of the first detection unit to an operating mode for detecting the state of the internal space, or a stop mode for stopping detection of the state of the internal space; and,
when the mode of the first detection unit is the operating mode, a decision of whether or not to control an operation of the inhalation device in accordance with a detection value detected by means of the first detection unit.
(2)

The inhalation device as disclosed in (1) above, further comprising a heating unit for heating a substrate accommodated in the accommodating portion, wherein
the operation of the inhalation device, for which the decision of whether or not to perform control, which is made by the control unit based on the detection result obtained by means of the second detection unit, is heating afforded by the heating unit.
(3)

The inhalation device as disclosed in (1) or (2) above,
further comprising a cover portion capable of opening/closing the opening leading to the internal space of the accommodating portion, wherein
the second detection unit detects at least any of: opening/closing of the opening by the cover portion, input of an instruction to start or stop detection by the first detection unit, or an automatic resolution-impossible error state, which is a state where an error relating to operation of the inhalation device has occurred, and the error cannot be automatically resolved by means of the control unit.
(4)

The inhalation device as disclosed in (3) above, wherein the control unit performs control to switch the mode of the first detection unit from the operating mode to the stop mode when the second detection unit has detected closure of the opening, input of an instruction to stop detection by the first detection unit, or an automatic resolution-impossible error state.
(5)

The inhalation device as disclosed in (4) above, comprising a plurality of first detection units, wherein
the control unit performs control to switch the mode of all of the plurality of first detection units which are in the operating mode to the stop mode when the second detection unit has detected closure of the opening, input of an instruction to stop detection by the first detection units, or an automatic resolution-impossible error state.
(6)

The inhalation device as disclosed in (5) above, wherein the control unit performs control to switch the mode of the first detection units from the stop mode to the operating mode when the second detection unit has detected opening of the opening, input of an instruction to start detection by the first detection units, or clearing of an automatic resolution-impossible error state.
(7)

The inhalation device as disclosed in (6) above, wherein the control unit performs control to switch the mode of only one of the plurality of first detection units from the stop mode to the operating mode when the second detection unit has detected opening of the opening, input of an instruction to start detection by the first detection unit, or clearing of an automatic resolution-impossible error state.
(8)

The inhalation device as disclosed in any one of (1) to (7) above, further comprising:
a heating unit for heating a substrate accommodated in the accommodating portion; and
a power source unit for storing power, wherein
the second detection unit detects at least any of: connection and disconnection of charging of the power source unit; input of an instruction to shift to a state in which heating by the heating unit can be implemented or is prohibited; the start or termination of switching, by means of the control unit, of a heating profile indicating a time-series transition of heating performed by the heating unit; the start or clearing of an automatic resolution error state, which is a state where an error relating to operation of the inhalation device has occurred, and the error can be automatically resolved by means of the control unit; or an instruction to cause the inhalation device to sleep or to cancel sleeping.
(9)

The inhalation device as disclosed in (8) above, wherein connection of charging of the power source unit, input of an instruction to shift to a state in which heating by the heating unit is prohibited, the start of switching of the heating profile by means of the control unit, the start of an automatic resolution error state, or an instruction to cause the inhalation device to sleep, constitutes a first operation;
disconnection of charging of the power source unit, input of an instruction to shift to a state in which heating by the heating unit can be implemented, termination of switching of the heating profile by means of the control unit, clearing of an automatic resolution error state, or an instruction to cancel sleeping of the inhalation device, constitutes a second operation; and
when the first operation has been detected by the second detection unit, the control unit decides that control of operation of the inhalation device based on the detection value detected by the first detection unit should not be performed until the second operation is detected.
(10)

The inhalation device as disclosed in (9) above, comprising a plurality of first detection units, wherein
the control unit controls the plurality of first detection units so that the mode of only one of the plurality of first detection units is the operating mode when the first operation has been detected by the second detection unit.
(11)

The inhalation device as disclosed in (9) or (10) above, wherein, when the first operation has been detected by means of the second detection unit during a period from detection by the first detection unit of a detection value exceeding a first threshold for determining insertion of the substrate until detection of a detection value falling below a second threshold for determining withdrawal of the substrate, the control unit decides that control of operation of the inhalation device based on the detection value detected by means of the first detection unit should not be performed until a detection value falling below the second threshold is detected by means of the first detection unit.
(12)

The inhalation device as disclosed in (11) above, wherein, when a detection value exceeding the first threshold has been detected during a period from detection of the first operation by the second detection unit until detection of the second operation by the second detection unit, the control unit decides that control of operation of the inhalation device based on the detection value detected by means of the first detection unit should not be performed until a detection value falling below the second threshold is detected by means of the first detection unit.
(13)

The inhalation device as disclosed in any one of (1) to (12) above, wherein the first detection unit detects the state of the internal space by emitting light into the internal space and detecting reflected light received.
(14)

The inhalation device as disclosed in any one of (1) to (13) above, further comprising a substrate accommodated in the accommodating portion.
(15)

An information processing method implemented by means of a computer for controlling an inhalation device comprising:
an accommodating portion having an internal space and an opening enabling the internal space to communicate with the outside;
a first detection unit for detecting a state of the internal space; and
a second detection unit for detecting information relating to a situation of the inhalation device,
   wherein
the information processing method comprises performing, based on a detection result obtained by means of the second detection unit:
control to switch a mode of the first detection unit to an operating mode for detecting the state of the internal space, or a stop mode for stopping detection of the state of the internal space; and,
when the mode of the first detection unit is the operating mode, a decision of whether or not to control an operation of the inhalation device in accordance with a detection value detected by means of the first detection unit.

### REFERENCE SIGNS LIST

100 Inhalation device
111 Power source unit
112 Sensor unit
113 Notification unit
114 Memory unit
115 Communication unit
116 Control unit
121 Heating unit
140 Accommodating portion
140A Stick lower portion accommodating portion
140B Guide portion
141 Internal space
142 Opening
143 Bottom portion
144 Heat insulating portion
150 Stick-type substrate
170 Light sensor unit
172 Circuit board
173 Light transmitting filter
174 Reinforcing plate
175 Clearance
176 Light-emitting unit
177 Light-receiving unit
178 Detection memory unit
179 Detection control unit
190 Cleaning article
191 Shaft portion
192 Cleaning portion

1-15. (canceled)

16. An inhalation device comprising:
an accommodating portion having an internal space and an opening enabling the internal space to communicate with the outside;
a first sensor configured to detect a state of the internal space;
a second sensor configured to detect information relating to a state of the inhalation device; and
processing circuitry configured to, based on a detection result obtained by the second sensor:
switch a mode of the first sensor to an operating mode for detecting the state of the internal space or to a stop mode for stopping the detecting of the state of the internal space; and
when the mode of the first sensor is the operating mode, determine whether or not to control a first operation of the inhalation device in accordance with a detection value detected by the first sensor.

17. The inhalation device as claimed in claim 16, further comprising a heater configured to heat a substrate accommodated in the accommodating portion,
wherein the first operation of the inhalation device is heating by the heater.

18. The inhalation device as claimed in claim 16, further comprising a cover portion capable of opening/closing an opening leading to the internal space of the accommodating portion,
wherein the second sensor is configured to detect at least one of:
the opening/closing of the opening by the cover portion,
input of an instruction to start or stop detection by the first sensor, or
an automatic resolution-impossible error state, which is a state where an error relating to a second operation of the inhalation device has occurred and the error cannot be automatically resolved by the processing circuitry.

19. The inhalation device as claimed in claim 18, wherein the processing circuitry is configured to switch the mode of the first sensor from the operating mode to the stop mode when the second sensor has detected the closing of the opening, the input of the instruction to stop detection, or the automatic resolution-impossible error state.

20. The inhalation device as claimed in claim 19, comprising a plurality of the first sensor,
wherein the processing circuitry is configured to switch the mode of all of the plurality of first sensors which are in the operating mode to the stop mode when the second sensor has detected the closing of the opening, the input of the instruction to stop detection, or the automatic resolution-impossible error state.

21. The inhalation device as claimed in claim 20, wherein the processing circuitry is configured to switch the mode of the plurality of first sensors from the stop mode to the operating mode when the second sensor has detected the opening of the opening, the input of the instruction to start detection, or a clearing of the automatic resolution-impossible error state.

22. The inhalation device as claimed in claim 21, wherein the processing circuitry is configured to switch the mode of only one of the plurality of first sensors from the stop mode to the operating mode when the second sensor has detected the opening of the opening, the input of the instruction to start detection, or the clearing of the automatic resolution-impossible error state.

23. The inhalation device as claimed in claim 16, further comprising:
a heater configured to heat a substrate accommodated in the accommodating portion; and
a power source configured to store power,
wherein the second sensor is configured to detect at least one of:
connection/disconnection of charging of the power source;
input of an instruction to shift to a state in which heating by the heater can be implemented or is prohibited;
start or termination of switching, by the processing circuitry, of a heating profile indicating a time-series transition of heating performed by the heater;
start or clearing of an automatic resolution error state, which is a state where an error relating to second operation of the inhalation device has occurred and the error can be automatically resolved by the processing circuitry; or
an instruction to cause the inhalation device to sleep or to cancel sleeping.

24. The inhalation device as claimed in claim 23, wherein
a third operation includes the connection of charging of the power source, the input of the instruction to shift to the state in which the heating by the heater is prohibited, the start of the switching of the heating profile by the processing circuitry, the start of the automatic resolution error state, or the instruction to cause the inhalation device to sleep;
a fourth operation includes the disconnection of charging of the power source, the input of the instruction to shift to a state in which the heating by the heater can be implemented, the termination of the switching of the heating profile by the processing circuitry, the clearing of the automatic resolution error state, or the instruction to cancel sleeping of the inhalation device; and
when the third operation has been detected by the second sensor, the processing circuitry is configured to determine that control of the first operation of the inhalation device in accordance with the detection value detected by the first sensor should not be performed until the fourth operation is detected.

25. The inhalation device as claimed in claim 24, comprising a plurality of the first sensor,
wherein the processing circuitry is configured to control the plurality of first sensors so that the mode of only one of the plurality of first sensors is the operating mode when the third operation has been detected by the second sensor.

26. The inhalation device as claimed in claim 24, wherein, when the third operation has been detected by the second sensor during a period from detection by the first sensor of a detection value exceeding a first threshold for determining insertion of the substrate until detection of a detection value falling below a second threshold for determining withdrawal of the substrate, the processing circuitry is configured to determine that control of the first operation of the inhalation device in accordance with the detection value detected by the first sensor should not be performed until a detection value falling below the second threshold is detected by the first sensor.

27. The inhalation device as claimed in claim 26, wherein, when a detection value exceeding the first threshold has been detected by the first sensor during a period from detection of the third operation by the second sensor until detection of the fourth operation by the second sensor, the processing circuitry is configured to determine that control of the first operation of the inhalation device in accordance with the detection value detected by the first sensor should not be performed until a detection value falling below the second threshold is detected by the first sensor.

28. The inhalation device as claimed in claim 16, wherein the first sensor is configured to detect the state of the internal space by emitting light into the internal space and detecting reflected light received.

29. The inhalation device as claimed in claim 16, further comprising a substrate accommodated in the accommodating portion.

30. The inhalation device as claimed in claim 16, wherein the second sensor is configured to accept an input from a user.

31. An information processing method for controlling an inhalation device, the method comprising:
performing, based on a detection result obtained by a second sensor configured to detect information relating to a state of the inhalation device,
switching a mode of a first sensor, configured to detect a state of an internal space of an accommodating portion having the internal space and an opening enabling the internal space to communicate with the outside, to an operating mode for detecting the state of the internal space or a stop mode for stopping the detecting of the state of the internal space; and
deciding, when the mode of the first sensor is the operating mode, whether or not to control an operation of the inhalation device in accordance with a detection value detected by the first sensor.

32. An inhalation device comprising:
an accommodating portion having an internal space and an opening enabling the internal space to communicate with the outside;
a first sensor configured to detect a state of the internal space;
a second sensor configured to detect information relating to a state of the inhalation device; and
processing circuitry configured to perform, based on a detection result obtained by the second sensor, a determination as to whether or not to control an operation of the inhalation device in accordance with a detection value detected by the first sensor.

33. The inhalation device as claimed in claim 32, further comprising a heater configured to heat a substrate accommodated in the accommodating portion,
wherein the operation of the inhalation device is heating by the heater.

34. The inhalation device as claimed in claim 32, wherein:
the second sensor is configured to detect a first operation and a second operation;
the first operation includes at least one of: connection of charging of a power source, input of an instruction to shift to a state in which heating by a heater is prohibited, a start of switching of a heating profile by the processing circuitry, a start of an automatic resolution error state, or an instruction to cause the inhalation device to sleep;
the second operation includes at least one of: disconnection of charging of the power source, input of an instruction to shift to a state in which heating by the heater can be implemented, termination of the switching of the heating profile by the processing circuitry, clearing of the automatic resolution error state, or an instruction to cancel sleeping of the inhalation device; and
when the first operation has been detected by the second sensor, the processing circuitry is configured to determine that control of the operation of the inhalation device in accordance with the detection value detected by the first sensor should not be performed until the second operation is detected.

35. The inhalation device as claimed in claim 34, wherein, when the first operation has been detected by the second sensor during a period from detection by the first sensor of a detection value exceeding a first threshold for determining insertion of a substrate until detection of a detection value falling below a second threshold for determining withdrawal of the substrate, the processing circuitry is configured to determine that control of the operation of the inhalation device in accordance with the detection value detected by the first sensor should not be performed until a detection value falling below the second threshold is detected by the first sensor.
